SEQUENCE LISTING (30 sequences)

SEQ ID NO: 1 - protein, 84 aa, Homo sapiens
MALSSQIWAACLLLLLLLASLTSGSVFPQQTGQLAELQPQDRAGARASWMPMFQRRRRRDTHFPICIFCCGCCHRSKCGMCCKT

SEQ ID NO: 2 - protein, 59 aa, Homo sapiens
VFPQQTGQLAELQPQDRAGARASWMPMFQRRRRRDTHFPICIFCCGCCHRSKCGMCCKT

SEQ ID NO: 3 - protein, 25 aa, Homo sapiens
DTHFPICIFCCGCCHRSKCGMCCKT

SEQ ID NO: 4 - protein, 22 aa, Homo sapiens
FPICIFCCGCCHRSKCGMCCKT

SEQ ID NO: 5 - protein, 20 aa, Homo sapiens
ICIFCCGCCHRSKCGMCCKT

SEQ ID NO: 6 - protein, 83 aa, Mus musculus
MALSTRTQAACLLLLLLASLSSTTYLHQQMRQTTELQPLHGEESRADIAIPMQKRRKRDTNFPICIFCCKCCNNSQCGICCKT

SEQ ID NO: 7 - protein, 25 aa, Mus musculus
DTNFPICIFCCKCCNNSQCGICCKT

SEQ ID NO: 8 - protein, 83 aa, Macaca fascicularis
MALSSQIWATCLLLLLLLASLTSGSVFPQQTGQLAELQPQDRAGARASWTPMLQRRRRRDTHFPICIFCCGCCHRSKCGMCCR

SEQ ID NO: 9 - protein, 24 aa, Macaca fascicularis
DTHFPICIFCCGCCHRSKCGMCCR

SEQ ID NO: 10 - protein, 84 aa, Macaca mulatta
MALSSQIWATCLLLLLLLASLTSGSVFPQQTGQLAELQPQDRAGARASWTPMLQRRRRRDTHFPICIFCCGCCHRSKCGMCCRT

SEQ ID NO: 11 - protein, 84 aa, Pan troglodytes
MALSSQIWAACLLLLLLLASLTSGSVFPQQTGQLAELQPQDRAGARASWMPMLQRRRRRDTHFPICIFCCGCCHRSKCGMCCKT

SEQ ID NO: 12 - protein, 84 aa, Pongo abelii
MALSSQIWAACLLLLLLLASLTSGSVFPQQTGQLAELQPQDRAGARAGWTPMLQRRRRRDTHFPIYIFCCGCCHRSKCGMCCKT

SEQ ID NO: 13 - protein, 85 aa, Nomascus leucogenys
MALSSQIWAACLLLLLLLASLTSGSVFPQQTGQLAELQGQLDRAGARAGWTPMLQRRRRRDTHFPICIFCCGCCHRSKCGMCCKT

SEQ ID NO: 14 - protein, 85 aa, Nomascus leucogenys; identical to SEQ ID NO: 13

SEQ ID NO: 15 - protein, 84 aa, Callithrix jacchus
MALSSQIWAVCLFLLLLLASLTSGFVFPQQAGQLTELQPQDRAGARASWMPMIQRRRRRDTHFPICIFCCGCCRQSNCGMCCKT

SEQ ID NO: 16 - protein, 83 aa, Ailuropoda melanoleuca
MALSTRIQAACLLSLLLASLASGSVFPHQTGQLAALQAQDAAGAEAGLMPALPRLRRRDTHFPICLFCCGCCNKSKCGICCKT

SEQ ID NO: 17 - protein, 82 aa, Bos taurus
MALNTQIRATCLLLLVLLSLTSGSVLPPQTRQLTDLQTKDTAGAAAGLTPVLQRRRRDTHFPICIFCCGCCRKGTCGMCCRT

SEQ ID NO: 18 - protein, 82 aa, Ovis aries
MALSTQTRATCLLLLVLLSLTSGSVLPPQTRQLTDLQTQHTAGAAAGLTPVLQRRRRDTHFPICIFCCGCCRKGTCGICCKT

SEQ ID NO: 19 - protein, 82 aa, Sus scrofa
MALSVQIRAACLLLLLLVSLTAGSVLPSQTRQLTDLRTQDTAGATAGLTPVAQRLRRDTHFPICIFCCGCCRKAICGMCCKT

SEQ ID NO: 20 - protein, 85 aa, Canis lupus familiaris
MALSTRIQAACLLLLLLASVASVSVLPHQTGQLTDLRAQDTAGAEAGLQPTLQLRRLRRRDTHFPICIFCCGCCKTPKCGLCCKT

SEQ ID NO: 21 - protein, 86 aa, Equus caballus
MALNTNIRAACLLLLLLASLTSGSVLPHQTRQLADLQTQDAAGMAGAAAGLMPGLHQLRRRDTHFPICTLCCGCCNKQKCGWCCKT

SEQ ID NO: 22 - protein, 84 aa, Rattus norvegicus
MALSTRIQAACLLLLLLASLSSGAYLRQQTRQTTALQPWHGAESKTDDSALLMLKRRKRDTNFPICLFCCKCCKNSSCGLCCIT

SEQ ID NO: 23 - protein, 84 aa, Mus musculus
MMALSTRTQAACLLLLLLASLSSTTYLQQQMRQTTELQPLHGEESRADIAIPMQKRRKRDINFPICRFCCQCCNKPSCGICCEE

SEQ ID NO: 24 - protein, 81 aa, Xenopus (Silurana) tropicalis
MKPVPICCLLLLLSFICHRGHSASLSGNEVTVTGNQIPETQMEESNALEPLLRSKRQSHLSICVHCCNCCKYKGCGKCCLT

SEQ ID NO: 25 - protein, 96 aa, Ictalurus punctatus
MRAMSIACAVAVIIACVCALQSAALPSEVRLDPEVRLEEPEDSEAARSIDQGVAAALAKETSPEVRFRTKRQSHLSLCRYCCNCCKNKGCGFCCRF

SEQ ID NO: 26 - protein, 91 aa, Danio rerio
MKLSNVFLAAVVILTCVCVFQITAVPFIQQVQDEHHVESEELQENQHLTEAEHRLTDPLVLFRTKRQSHLSLCRFCCKCCRNKGCGYCCKF

SEQ ID NO: 27 - protein, 91 aa, Danio rerio
MKLSNVFLAAVVILTCVCVFQITAVPFIQQVQDEHHVESEELQENQHLTEAEHRLADPLVLFRTKRQSHLSLCRFCCKCCRNKGCGYCCKF

SEQ ID NO: 28 - protein, 96 aa, Ictalurus punctatus
MRPMSIACAVAVIIACVCALQSAALPSEVRLDPEVRLEEPEDSEAARSIDQGVAAALAKETSPEVLFRTKRQSHLSLCRYCCNCCKNKGCGFCCRF

SEQ ID NO: 29 - protein, 80 aa, Xenopus tropicalis
MKSLLLCCLLLLLSLICHRGHSASLSGNEIKAPEHPISESEQGESDALGPLFRTKRHLNICVYCCKCCKKQKGCGMCCFT

SEQ ID NO: 30 - protein, 88 aa, Salmo salar
MKAFSVAVAVVVVLACMFILESTAVPFSEVRTEEVESIDSPVGEHQQPGGTSMNLPMHFRFKRQSHLSLCRWCCNCCHNKGCGFCCKF

(19) United States
(12) Patent Application Publication
(10) Pub. No.: US 2013/0236977 A1
(43) Pub. Date: Sep. 12, 2013

(54) COMPOSITIONS AND METHODS FOR PLASMA PEPTIDE ANALYSIS

(71) Applicants: Hanno Steen, Cambridge, MA (US); Damon Anderson, Wexford, PA (US)

(72) Inventors: Hanno Steen, Cambridge, MA (US); Damon Anderson, Wexford, PA (US)

(73) Assignee: Children's Medical Center Corporation, Boston, MA (US)

(21) Appl. No.: 13/699,836

(22) PCT Filed: May 24, 2011
(86) PCT No.: PCT/US11/37749

§ 371 Date: May 28, 2013

Related U.S. Application Data
(60) Provisional application No. 61/347,630, filed on May 24, 2010.

(51) Int. Cl. G01N 33/74; G01N 33/68

(52) U.S. Cl. CPC G01N 33/74; G01N 33/6851
USPC 436/86; 530/324; 422/430

(57) ABSTRACT

Provided herein are composition, methods and kits for peptide analysis in blood by mass spectroscopy. Isotopic peptides are also provided that facilitate quantification of peptide levels, e.g., hepcidin levels, in blood by mass spectroscopy. Further disclosed are methods and compositions for quantifying blood hepcidin levels and evaluating iron-associated disorders.

### RELATED APPLICATIONS

This application claims the benefit under 35 U.S.C. §119(e) of U.S. Provisional Application Ser. No. 61/347,630, entitled “COMPOSITIONS AND METHODS FOR PLASMA PEPTIDE ANALYSIS”, filed on May 24, 2010, the entire disclosure of which is herein incorporated by reference in its entirety.

### GOVERNMENT FUNDING

This invention was made with United States Government support under grant T32 HL076115 awarded by the National Institutes of Health. The United States government has certain rights in the invention

### FIELD OF THE INVENTION

The invention relates to composition and methods for analyzing peptides in blood by mass spectroscopy.

### BACKGROUND OF INVENTION

Iron deficiency anemia (IDA) is the most common nutritional deficiency worldwide. In the United States, IDA affects nearly 3% of 1-2 year old children and at least 9% more are iron deficient (ID) and are at risk for neurodevelopmental defects. Recommendations vary, but consensus indicates that pediatricians should screen infants for iron deficiency within the first year of life. Screening methods also vary, as a balance must be made between sensitivity, specificity, rapidity, invasiveness, and cost. The most sensitive test, the cellular hemoglobin of the reticylocytes (CHr), is the most invasive, most costly, and least available in the clinic. The least costly, least invasive test, the finger stick hemoglobin, is also the least sensitive. Moreover, though it is imperative to detect early iron deficiency before anemia develops, many screening tests do not directly measure the patient iron status.

Recently an iron regulatory hormone hepcidin has been identified. Hepcidin (HepC-25), a 25 amino acid circulatory peptide produced in the liver, functions by binding to the sole iron membrane protein ferroportin on the surface of intestinal enterocytes, hepatocytes, or macrophages, causing its internalization and degradation. In doing so, HepC-25 regulates iron influx from the intestinal deodenum, iron release from intracellular storage sites within hepatocytes and recycling macrophages. Consequently, elevated HepC-25 levels serve to down regulate dietary iron uptake and suppress iron mobilization through the reticulocyte system, while low HepC-25 levels serve to stimulate iron absorption and erythropoiesis.

Hepcidin was initially identified in 2001 during the search for novel anti-microbial peptides in urine and blood ultra-filtrates. Subsequent mouse genetic studies affirmed the role of HepC-25 as the major regulator of iron transport in mammalian systems. It is now known that altered HepC-25 production is associated with a number of pathological conditions; decreased HepC-25 levels are linked to hereditary hemochromatosis and iron deficiency anemia, while elevated HepC-25 levels are associated with iron refractory anemia, β-thalassemia, and anemia of chronic disease. Collectively, iron associated disorders affect over a billion people worldwide. Traditional immunoassay approaches for the quantification of hepcidin have been hampered by the difficulties associated generating specific high affinity antibodies to the human form of hepcidin. Hepcidin is highly conserved among mammalian species, differing by only a few n-terminal residues, thus it is a difficult peptide upon which to generate immunoreactivity and specificity. Nonetheless, several hepcidin immunoassays have been designed (Intrinsic Life Sciences) (22, 23). The ability to measure circulating levels of HepC-25 offers diagnostic potential for the assessment and treatment of a number of important iron associated disorders. Yet, despite its importance, there is currently no clinically validated assay for hepcidin in human plasma.

The clinical diagnostics landscape is populated by a number of standardized analytical platforms, including immunological, spectrophotemetric, fluorometric and radiological type assays. The main advantages of these testing platforms include the relative simplicity and common availability of reagents and instrumentation making widespread adoption of standardized tests feasible. A limitation of these off-the-shelf methodologies is the inability to perform true discovery based research and assay development, as these methods have a general requirement for a known biologically relevant target or analyte around which the detection reagent (antibody or fluorophorogenic substrate) is based. In contrast, state-of-the-art technologies including modern biological mass spectrometry (MS) offer both ultra sensitive detection capabilities and the opportunity to perform discovery oriented research when coupled with the appropriate analytical components. For instance a typical linear triple quadrupole mass spectrometer is capable of sub-attamole sensitivity and extremely high accuracy detection of small molecule metabolites and peptide hormones. When coupled with a liquid chromatographic interface upstream of the MS, protein or peptide profiling may be performed from biological mixtures. In some instances, sequencing and quantification of molecules of interest is possible, through the use of an in-source/post-source collision cell.

Despite the advantages of MS, the technology as a whole has been largely limited to the basic research realm, partly a result of the complex instrumentation and relatively low-throughput workflows associated with MS techniques (for review (1, 2)). MS has been implemented in the clinical labs for the detection of small molecules, i.e., testosterone, caffeine, vitamin D; however, larger peptides and proteins have been excluded from clinical method development primarily for logistical reasons. Prototypical liquid chromatography mass spectrometry (LC-MS) based analysis demands both efficient analyte ionization (free from background matrix effects), and effective peptide/protein fragmentation in order to gain quantitative information based on peptide/protein identity. Therefore, rather extensive sample preparation is usually involved, thereby complicating the workflow and extending processing times. Modern techniques have sought to address these limitations, including those involving tools such as multiple-reaction monitoring (MRM) (3) or multiplexed LC column switching approaches, but these have yet to find their way into the clinical labs.

### SUMMARY OF INVENTION

Aspects of the invention relate to methods, compositions and kits for processing biological samples for peptide analysis by mass spectroscopy. In some aspects of the invention, preparative methods are provided that facilitate reliable, reproducible and highly sensitive detection and quantification of peptide levels in blood. Methods of the invention are amenable to use in a high-throughput context, and accordingly, enable multiplex peptide analysis. Methods of the invention are useful in a variety of different settings including, but not limited to, basic science research, pharmaceutical discovery and development, and clinical diagnosis. In certain aspects of the invention, methods, compositions and kits are provided that facilitate the detection and quantification of hepcidin levels in blood samples by mass spectroscopy. Isotopic peptide standards are also provided. In further aspects of the invention, methods are provided for evaluating iron-associated disorders in a subject based on hepcidin levels. Accordingly, methods for diagnosing iron-associated disorders in a subject based on hepcidin levels are also provided.

According to some aspects of the invention, methods are provided for detecting a peptide in a subject. In some embodiments, the methods comprise: (a) processing a blood sample obtained from a subject by: (i.) combining the blood sample with a denaturation buffer comprising a volatile organic acid, in which the concentration of the volatile organic acid in the combination is between 1% and 50%; and (ii.) separating a soluble fraction from an insoluble fraction of the combination; and (b) performing MALDI-TOF mass spectrometry on the soluble fraction to detect the peptide. In some embodiments, the concentration of the volatile organic acid in the combination is 1%. In some embodiments, the concentration of the volatile organic acid in the combination is 4%. In certain embodiments, the volatile organic acid is selected from the group consisting of: a citric acid, a cyanic acid, a lactic acid, a formic acid, an acetic acid, a propionic acid, and a butyric acid. In certain embodiments, the volatile organic acid is trifluoroacetic acid (TFA). In some embodiments, the denaturation buffer further comprises an organic solvent. In certain embodiments, the concentration of the organic solvent in the combination is up to 40%. In certain embodiments, the concentration of the organic solvent in the combination is 10%. In certain embodiments, the concentration of the organic solvent in the combination is 20%. In certain embodiments, the organic solvent is selected from the group consisting of: acetonitrile, acetone, isopropanol, ethanol, methanol, or a combination thereof. In some embodiments, processing the test sample further comprises adding a pre-treatment solution comprising a surfactant to the blood sample prior to step (i.). In certain embodiments, the surfactant is a non-ionic surfactant selected from the group consisting of: Triton X-45, Triton X-100, Triton X-114 Triton X-165, Triton X-305, Triton X-405, Triton X 705-70, Triton CF10, Tween 20, Tween-80, sodium cholate, β-propriolactone, Octyl glucoside, Decyl maltoside and polyoxyethylene-20 sorbitan monooleate. In some embodiments, the final concentration of the pre-treatment solution in the blood sample is in a range 0.1% to 5%. In some embodiments, the pretreatment solution further comprises an organophosphate. In certain embodiments, the organophosphate is tri-n-butyl phosphate. In some embodiments, separating the soluble fraction in step (ii.) comprises filtering the combination and extracting filtrate. In some embodiments, separating the soluble fraction in step (ii.) comprises centrifuging the combination and extracting supernatant. In some embodiments, the peptide has a molecular weight in a range of 0.3 kDa to 10 kDa.

In some embodiments, the peptide is hepcidin. In some embodiments, the hepcidin has an amino acid sequence as set forth in any one of SEQ ID NO: 1 to 30. In some embodiments, hepcidin has an amino acid sequence as set forth in SEQ ID NO: 3, 4, 5, 7 or 9. In some embodiments, hepcidin has an amino acid sequence consisting of the portion of an amino sequence as set forth in any one of SEQ ID NO: 1, 2, 6, 8, and 10-30 that corresponds to mature hepcidin. In some embodiments, hepcidin has the amino acid sequence of the last 25 amino acids at the C-terminus of an amino sequence as set forth in any one of SEQ ID NO: 1, 2, 6, 8, or 10-30.

In some embodiments, the subject is a vertebrate. In some embodiments, the subject is a human. In some embodiments, the subject is a non-human primate. In some embodiments, the subject is a monkey (e.g., a cynomolgus monkey). In some embodiments, the subject is a mouse.

In some embodiments, the methods comprise: (a) processing a blood sample obtained from a subject by enriching the sample for hepcidin and (b) performing MALDI-TOF mass spectrometry on the enriched sample to detect the hepcidin. In some embodiments, the enrichment step comprises contacting the sample with a weak cation exchange resin (e.g., silica and/or agarose). In one embodiment, the resin is washed with a low salt aqueous buffer (e.g., 0.1-1% KCl). In some embodiments, the sample is eluted from the resin with an acidic aqueous buffer (e.g., 0.5-5% TFA with 0.1-10% ACN). In some embodiments, the enrichment step comprises contacting the sample with a reverse phase resin (e.g., silica and/or agarose). In one embodiment, the resin is washed with a mild organic buffer (e.g., 0.1-1.0% ACN). In one embodiment, the sample is eluted from the resin with a high organic buffer (e.g., 50-70% ACN). In one embodiment, the eluate is subjected to conditions that promote evaporation of the organic buffer to below a predetermined threshold level (e.g., from 50-70% ACN to less than 40% ACN) and/or to complete dryness. In some embodiments, after evaporation, the eluate is reconstituted in an aqueous buffer (e.g., 0.1-1% TFA, 0.1-5% ACN). In some embodiments, isotopic peptides are added to the sample at a known concentration prior to the enrichment step.

In some embodiments, the methods further comprise diluting the blood sample in water. In some embodiments, the blood sample is a serum sample. In some embodiments, the blood sample is a plasma sample. In some embodiments, the plasma sample comprises heparinized-plasma or EDTA-treated plasma. In some embodiments, prior to step (a) the blood sample is thawed. In some embodiments, the blood sample is thawed following storage at −80° C.

According to some aspects of the invention, methods for detecting a peptide in a plurality of subjects are provided. In some embodiments, the methods comprise implementing any of the foregoing methods for detecting a peptide in a subject in a multiplex format to detect the peptide in blood samples obtained from a plurality of subjects.

According to some aspects of the invention, methods are provided for determining hepcidin levels in a subject. In some embodiments, the methods comprise: (a) adding a known quantity of isotopic hepcidin to a blood sample obtained from a subject, wherein the isotopic hepcidin has a molecular weight in a range of 8 Da to 12 Da greater than the molecular weight of hepcidin, (b) detecting hepcidin and isotopic hepcidin in the blood sample by performing mass spectroscopy on the blood sample after step (a), and (c) comparing the hepcidin and isotopic hepcidin detected in (b). In some embodiments, the methods further comprise: (d) determining hepcidin levels in the subject based on the comparison in (c). In certain embodiments, the hepcidin levels are indicative of iron levels in the subject. In certain embodiments, the hepcidin levels are indicative of an iron-associated disorder in the subject. In some embodiments, the methods further comprise: diagnosing the subject as having the iron-associated disorder, wherein the diagnosis is based, at least in part, on the hepcidin levels. In some embodiments, significantly higher hepcidin levels in the subject compared with a normal subject indicate a diagnosis of anemia of chronic disease (anemia of inflammation), iron-refractory iron deficiency anemia, rheumatologic disease, inflammatory bowel disease, multiple myeloma, or cancer. In some embodiments, significantly lower hepcidin levels in the subject compared with a normal subject indicate a diagnosis of hereditary hemochromatosis, β-thalassemia, congenital hypoplastic anemia, hemolytic anemia, hereditary sideroachrestic anemia, hereditary sideroblastic anemia, microcytic anemia, macrocytic anemia, megaloblastic anemia, sickle cell anemia or another iron loading anemia. In some embodiments, detecting hepcidin and isotopic hepcidin in (b) comprises: (A.) processing the blood sample of step (a) by: (i.) combining the blood sample with a denaturation buffer comprising a volatile organic acid, wherein the concentration of the volatile organic acid in the combination is between 1% and 50%; and (ii.) separating a soluble fraction from an insoluble fraction of the combination; and (B.) performing MALDI-TOF mass spectrometry on the soluble fraction to detect the hepcidin and isotopic hepcidin. In certain embodiments, the concentration of the volatile organic acid in the combination is 1%. In certain embodiments, the concentration of the volatile organic acid in the combination is 4%. In some embodiments, the volatile organic acid is selected from the group consisting of: a citric acid, a cyanic acid, a lactic acid, a formic acid, an acetic acid, a propionic acid, and a butyric acid. In some embodiments, the volatile organic acid is trifluoroacetic acid (TFA). In some embodiments, the denaturation buffer further comprises an organic solvent. In certain embodiments, the concentration of the organic solvent in the combination is up to 40%. In certain embodiments, the concentration of the organic solvent in the combination is 10%. In certain embodiments, the concentration of the organic solvent in the combination is 20%. In certain embodiments, the organic solvent is selected from the group consisting of: acetonitrile, acetone, isopropanol, ethanol, methanol, or a combination thereof. In some embodiments, processing the test sample further comprises adding a pre-treatment solution comprising a surfactant to the blood sample prior to step (i.). In some embodiments, the surfactant is a non-ionic surfactant selected from the group consisting of: Triton X-45, Triton X-100, Triton X-114 Triton X-165, Triton X-305, Triton X-405, Triton X 705-70, Triton CF10, Tween 20, Tween-80, sodium cholate, β-propriolactone, and polyoxyethylene-20 sorbitan monooleate. In some embodiments, the final concentration of the pre-treatment solution in the blood sample is in a range 0.1% to 5%. In certain embodiments, the pretreatment solution further comprises tri-n-butyl phosphate. In some embodiments, separating the soluble fraction in step (ii.) comprises filtering the combination and extracting filtrate. In some embodiments, separating the soluble fraction in step (ii.) comprises centrifuging the combination and extracting supernatant. In some embodiments, the methods further comprise diluting the blood sample in water. In certain embodiments, the blood sample is a serum sample. In certain embodiments, the blood sample is a plasma sample. In some embodiments, the plasma sample comprises heparinized-plasma or EDTA-treated plasma. In certain embodiments, the hepcidin has an amino acid sequence as set forth in any one of SEQ ID NO: 1 to 30 or the portion of the sequence of any one of SEQ ID NO: 1, 6, 8, and 10-30 that corresponds to mature hepcidin.

According to some aspects of the invention, an isotopic hepcidin is provided that has an amino acid sequence as set forth in SEQ ID NO: 3, wherein the arginine at amino acid position 16 is arginine-¹⁵N₄¹³C₆. In some aspects of the invention, a composition comprising the isotopic hepcidin is provided. In some embodiments, the purity of the isotopic hepcidin in the composition is greater than or equal to 95%. In some embodiments, the purity of the isotopic hepcidin in the composition is greater than or equal to 99%. In some aspects of the invention, a kit comprising a container housing the isotopic hepcidin is provided. In some aspects of the invention, a kit comprising a container housing the composition is provided.

According to some aspects of the invention, a plate is provided that comprises a plurality of wells, each well comprising isotopic peptide having a molecular weight in a range of 8 Da to 12 Da greater than the molecular weight of a corresponding non-isotopic peptide, wherein the peptide has a molecular weight in a range of 0.2 kDa to 10 kDa. In some embodiments, the isotopic peptide is isotopic hepcidin and wherein the corresponding non-isotopic peptide has an amino acid sequence as set forth in any one of SEQ ID NO: 1 to 30 or the portion of the sequence of any one of SEQ ID NO: 1, 6, 8, and 10-30 that corresponds to mature hepcidin. In some embodiments, each well comprises a composition comprising the isotopic peptide and a volatile organic acid at a concentration in a range of 0.1% to 0.5%. In some embodiments, each well comprises a composition comprising the isotopic hepcidin and an organic solvent at a concentration up to 10%. In some embodiments, each well comprises a composition comprising the isotopic hepcidin, a volatile organic acid at a concentration in a range of 0.1% to 0.5% and an organic solvent at a concentration up to 10%. In some embodiments, each well comprising a composition comprising a volatile organic acid at a concentration in a range of 2% to 16%. In some embodiments, the composition further comprises an organic solvent at a concentration in a range of 20% to 80%. In some embodiments, each well comprises two chambers separated by a filter membrane, wherein the composition is disposed on the filter membrane in the first chamber. In some embodiments, the filter membrane is polyvinylidene fluoride (PVDF), polyethylene sulfone (PES), polycarbonate, polytetrafluoroethylene (PTFE), or glass fiber. In some embodiments, the filter membrane has a pore size in a range of 0.2 μm to 1.0 μm. In some embodiments, the volatile organic acid is selected from the group consisting of: a citric acid, a cyanic acid, a lactic acid, a formic acid, an acetic acid, a propionic acid, and a butyric acid. In some embodiments, the volatile organic acid is trifluoroacetic acid (TFA). In some embodiments, the organic solvent is selected from the group consisting of: acetonitrile, acetone, isopropanol, ethanol, methanol, or a combination thereof. In some embodiments, the composition further comprises a surfactant. In some embodiments, the surfactant is a non-ionic surfactant selected from the group consisting of: Triton X-45, Triton X-100, Triton X-114 Triton X-165, Triton X-305, Triton X-405, Triton X 705-70, Triton CF10, Tween 20, Tween-80, sodium cholate, β-propriolactone, and polyoxyethylene-20 sorbitan monooleate. In some embodiments, the concentration of surfactant in the composition is in a range 0.25% to 5%. In some embodiments, the composition further comprises an organophosphate. In some embodiments, the concentration of the organophosphate in the composition is in a range of 0.25% to 5%. In some embodiments, the organophosphate is tri-n-butyl phosphate.

According to some aspects of the invention, a kit is provided comprising any of the foregoing plates. In some embodiments, the kit further comprises a container housing isotopic hepcidin. In some embodiments, the kit further comprises a container housing a composition comprising isotopic hepcidin. In some embodiments, the kit further comprises a chip comprising an array of vacuum sublimated matrix spots for MALDI-TOF mass spectroscopy sample preparation. In some embodiments, the vacuum sublimated matrix spots comprise a crystallized molecule selected from the group consisting of: 3,5-dimethoxy-4-hydroxycinnamic acid (sinapinic acid or SA), α-cyano-4-hydroxycinnamic acid (alpha-cyano or alpha-matrix, or CHCA or HCCA) and 2,5-dihydroxybenzoic acid (DHB). In some embodiments, matrix spots are deposited on MALDI chips using methods that involve vacuum sublimation. In some embodiments, matrix spots are deposited on MALDI chips using methods that do not involve vacuum sublimation. In one embodiment, the MALDI chips are prepared using photolithography coupled with ink jet deposition of matrix on stainless steel targets (e.g., Hudson Surface Technology). It should be appreciated that prespotting of matrix may be accomplished by various techniques. In one embodiment, the matrix spots are surrounded by a hydrophobic mask (e.g., an ultrahydrophobic mask). In some embodiments, the hydrophobic mask permits focusing of aqueous droplets to facilitate analyte contact with the matrix and sample enrichment (e.g., Bruker anchorchips). Other approaches to matrix deposition will be apparent to the skilled artisan.

According to some aspects of the invention, a kit is provided that comprises a container housing an isotopic peptide and (a) a container housing a denaturation buffer comprising: (i.) a volatile organic acid, and/or (ii.) an organic solvent; and/or (b) a container housing a pre-treatment solution comprising a surfactant. In some embodiments, the isotopic peptide has a molecular weight in a range of 0.3 kDa to 10 kDa. In some embodiments, the same container houses the denaturation buffer and the pre-treatment solution. In certain embodiments, the volatile organic acid is selected from the group consisting of: a citric acid, a cyanic acid, a lactic acid, a formic acid, an acetic acid, a propionic acid, and a butyric acid. In certain embodiments, the volatile organic acid is trifluoroacetic acid (TFA). In certain embodiments, the organic solvent is selected from the group consisting of: acetonitrile, acetone, isopropanol, ethanol, methanol, or a combination thereof. In certain embodiments, the surfactant is a non-ionic surfactant selected from the group consisting of: Triton X-45, Triton X-100, Triton X-114 Triton X-165, Triton X-305, Triton X-405, Triton X 705-70, Triton CF10, Tween 20, Tween-80, sodium cholate, β-propriolactone, and polyoxyethylene-20 sorbitan monooleate. In some embodiments, the pretreatment solution further comprises an organophosphate. In certain embodiments, the organophosphate is tri-n-butyl phosphate. In some embodiments, the isotopic peptide is isotopic hepcidin. In certain embodiments, the isotopic hepcidin has an amino acid sequence as set forth in any one of SEQ ID NO: 1 to 30 or the portion of the sequence of any one of SEQ ID NO: 1, 6, 8, and 10-30 that corresponds to mature hepcidin. In certain embodiments, the isotopic hepcidin has an amino acid sequence as set forth in SEQ ID NO: 3 in which the arginine at amino acid position 16 is arginine-¹⁵N₄¹³C₆. In certain embodiments, the isotopic hepcidin has an amino acid sequence as set forth in SEQ ID NO: 7 in which the phenylalanine at amino acid position 4 or 9 is phenylalanine-¹⁵N¹³C. In certain embodiments, the isotopic hepcidin has an amino acid sequence as set forth in SEQ ID NO: 9 in which the phenylalanine at amino acid position 4 or 9 is phenylalanine-¹⁵N¹³C.

According to some aspects of the invention, a composition is provided that comprises a volatile organic acid at a concentration in a range of 1% to 8% and an organic solvent at a concentration in a range of 5% to 20%. In some embodiments, the composition further comprises an organophosphate at a concentration in a range of 0.25% to 2%. In some embodiments, the composition further comprises a surfactant in a range of 0.5% to 1%. In some embodiments, the composition further comprises an isotopic peptide having a molecular weight in a range of 0.2 kDa to 10 kDa. In certain embodiments, the isotopic peptide is isotopic hepcidin. In some embodiments, the composition further comprises a blood sample. In some embodiments, the blood sample is at concentration in a range of 30% to 60%. In certain embodiments, the blood sample is a serum sample. In certain embodiments, the blood sample is a plasma sample. In certain embodiments, the plasma sample comprises heparinized-plasma or EDTA-treated plasma. In some embodiments, the volatile organic acid is selected from the group consisting of: a citric acid, a cyanic acid, a lactic acid, a formic acid, an acetic acid, a propionic acid, and a butyric acid. In certain embodiments, the volatile organic acid is trifluoroacetic acid (TFA). In some embodiments, the concentration of the volatile organic acid is 4%. In some embodiments, the organic solvent is selected from the group consisting of:

acetonitrile, acetone, isopropanol, ethanol, methanol, or a combination thereof. In some embodiments, the concentration of the organic solvent is 10%. In certain embodiments, the surfactant is a non-ionic surfactant selected from the group consisting of: Triton X-45, Triton X-100, Triton X-114 Triton X-165, Triton X-305, Triton X-405, Triton X 705-70, Triton CF10, Tween 20, Tween-80, sodium cholate, β-propriolactone, and polyoxyethylene-20 sorbitan monooleate. In some embodiments, the concentration of the surfactant is in a range of 0.5% to 1%. In some embodiments, the organophosphate is tri-n-butyl phosphate. In some embodiments, the concentration of the organophosphate is in a range of 0.5% to 1%. According to some aspects of the invention, a plate is provided that comprises a plurality of wells, each well comprising any of the foregoing compositions.

### BRIEF DESCRIPTION OF DRAWINGS

FIG. 1A-B depicts the linear range and analytical sensitivity of hepcidin in plasma. Blank plasma was spiked with a dilution series of synthetic HepC-25 and a single concentration of stable isotope labeled synthetic HepC-25 internal standard.

FIG. 2 shows an analysis of preanalytical factors on hepcidin detection. FIG. 2A shows matched heparin plasma and EDTA plasma from a set of six healthy adults (2 females, 4 males, age 23-35, median 27). FIG. 2B shows spectra from matched samples. FIGS. 2C-D show hepcidin levels determined from heparin plasma and EDTA plasma samples (1-6) that were subjected to consecutive three-day freeze (−80° C.)/thaw (+25° C.) treatments. Bars show, left-to-right, results from day 0 through day 3. FIGS. 2E-F show hepcidin levels determined in heparin plasma and EDTA plasma that was stored at −80° C. Bars show, left-to-right, results from day 0, day 1, week 1, and month 1.

FIG. 3 shows a comparison between the plasma mass spectroscopy assay described in Example 2 and a previously described immunoassay. Samples (n=87) are expressed in ng/mL and plotted in linear scale.

FIG. 4 shows stability of hepcidin in platelet depleted plasma subjected to various temperature storage conditions (Sample 1—Na:Heparin, Sample 2—Na:Citrate, Sample 3—K₂:EDTA, Sample 4—K₂:EDTA P800, Sample 5—K₂:EDTA P100). Hepcidin was measured at Day 0, Day 1, Week 1, Month 1 (bars, left to right). FIGS. 4A, 4C, and 4E show the results of different storages temperatures. FIGS. 4B, 4D, and 4F show signal-to-noise values for Hepcidin isotopic envelopes monitored over the temperature time course.

FIG. 5A shows hepcidin levels in normal patient cohorts. (5A box 1. Pediatric normal cohort hepcidin mean 11.58 ng/mL; median 2 yrs; range 0.2 to 14 yrs; n=126. 5A box 2. Adult normal hepcidin mean 16.82 ng/mL; median 49 yrs; range 32 to 73 yrs; n=100. Difference (P =0.002).) FIG. 5B shows results from a peptide analysis of an Iron Refactory Iron Deficiency Anemia cohort (IRIDA). Use of the iron index (transferrin saturation (%)/(log₁₀) hepcidin (ng/mL)) enabled the differentiation between homozygous nulls (+/+)(FIG. 5B box 1.) and heterozygous (+/−)(P=0.006)(5B box 2.) homozygous (−/−)(P=0.0001)(5B box 3.) IRIDA mutations.

FIG. 6 provides a schematic of a non-limiting example of a work-flow for hepcidin analysis.

### DETAILED DESCRIPTION OF INVENTION

Aspects of the invention relate to methods and compositions for processing blood samples for peptide analysis by mass spectroscopy. In some aspects, the invention provides reliable, reproducible, and highly sensitive mass spectrometric based assays for detecting and quantifying peptides in blood. Isotopic peptide standards are provided that facilitate quantification of peptides in blood using the mass spectroscopic based methods. Further aspects of the invention provide an analytical assay for the detection and quantification of the peptide hormone hepcidin derived from human blood. Hepcidin is a small (e.g., 25-amino acid; 2789 Da) cationic (pI 10) liver-secreted circulating peptide that functions as the systemic regulator of iron status in mammals (5, 6). Hepcidin acts to regulate iron metabolism by binding directly to the transmembrane iron exporter, ferroportin, on the surface of intestinal enterocytes and macrophages, causing its internalization and degradation (7, 8). In this manner, hepcidin controls iron mobilization in the body by directly modulating iron uptake from the duodenum and iron release from macrophage intracellular storage sites. Typically, hepcidin has a beta-sheet rich structure with 4 disulfide bonds.

The term “hepcidin,” as used herein, refers to a preprohormone, prohormone or hormone form of hepcidin protein. In humans, hepcidin preprohormone, prohormone and hormone size are typically 84, 60 and 25 amino acids respectively; 20 and 22 amino acid forms of hepcidin also exist. The N terminal region of the mature hepcidin is important for function, and deletion of 5 N-terminal amino acids in humans may result in a loss of function. The hormone form of hepcidin (e.g., the 25 amino acid form of human hepcidin) may be referred to as “Hep-C”, “Hep-C-25”, or “mature hepcidin”. Exemplary amino acid sequences for hepcidin are provided in SEQ ID NO: 1 to 30. The amino acid sequences set forth in SEQ ID NO: 3, 7 and 9 are examples of mature hepcidin sequences for Homo sapiens, Mus musculus, and Macaca fascicularis, respectively. Examples of preprohormone hepcidin sequences for various species are set forth in SEQ ID NO: 1, 6, 8, and 10-30. The amino acid sequence of the mature hepcidin portion of these preprohormone proteins will be apparent to the skilled artisan. In some embodiments, the sequence of the mature hepcidin corresponds to the last 25 amino acids at the C-terminus of the preprohormone. In some embodiments, the mature hepcidin sequence has a tripeptide motif of DTH, DTN or DIH at its N-terminus. In some embodiments, mature hepcidin consists of the amino acid sequence of the last 30, 29, 28, 27, 26, 25, 24, 23, 22, 21, 20, or 19 amino acids at the C-terminus of an amino sequence as set forth in any one of SEQ ID NO: 1, 2, 6, 8, or 10-30.

Due to its central role in iron metabolism, hepcidin has been linked to a number of important iron-associated disorders. For example, increased hepcidin levels are associated with anemia of inflammation (9, 10) and iron-refractory iron deficiency anemia (11-13), whereas hepcidin deficiency is linked to most forms of hereditary hemochromatosis (14, 15), β-thalassemia, and other iron loading anemias (16). Furthermore, hepcidin levels correlate with altered iron status in a number of conditions including dietary iron deficiency anemia, hypoxia, liver cirhosis, cancer, and infection (17-21). Iron-associated disorders affect nearly 1 billion humans worldwide. In aspects of the invention, methods are provided for evaluating iron-associated disorders in a subject based on hepcidin levels. Accordingly methods for diagnosing iron-associated disorders in a subject based on hepcidin levels are also provided. Methods for monitoring the status of iron-associated disorders in a subject based on hepcidin levels are also provided.

### Methods for Peptide Analysis by Mass Spectroscopy

### Preparative Methods

Preparative methods are provided for processing blood samples to facilitate quantification and detection of peptide levels by mass spectroscopy. The methods involve processing a blood sample obtained from a subject by combining the blood sample with one or more different solutions in order to prepare a test sample suitable for mass spectroscopic analysis. The methods are useful for detecting low molecular weight peptides, e.g., peptides having a molecular weight in a range of 0.3 kDa to 10 kDa, such as hepcidin.

As used herein the term “blood sample” refers to blood obtained from a subject. A blood sample may be a whole-blood sample obtained from a subject, or a fraction thereof, e.g., a plasma sample, a serum sample, etc. A blood sample may be a whole-blood sample, or fraction thereof, that has been combined with water or a solution comprising an anticoagulant. The term “subject” as used herein refers to human and non-human animals. Typically, the term subject refers to a vertebrate, e.g., a mammal, such as a human or non-human primate, sheep, dog, rodent (e.g., mouse, rat), guinea pig, goat, pig, cat, rabbit, cow, and non-mammal such as a bird, amphibian, reptile, etc. In one embodiment, the subject is human. In another embodiment, the subject is a laboratory animal. In one embodiment, the subject has or is suspected of having an iron-associated disorder. In one embodiment, the subject is an animal model of an iron-associated disorder.

The blood sample may be obtained from a subject using methods well known in the art. As used herein, the phrase “obtaining a blood sample” refers to any process for directly or indirectly acquiring a blood sample from a subject. For example, a blood sample may be “obtained from a subject” by procuring (e.g., at a point-of-care facility, e.g., a physician's office, a hospital) a blood sample directly from the subject (e.g., by a venous blood draw). Alternatively, a blood sample may be “obtained from a subject” by receiving the blood sample (e.g., at a laboratory facility) from one or more persons who procured the blood sample directly from the subject. It is often desirable to use venous blood drawn from a subject in a clinical setting. A blood sample may or may not be collected from a subject who has been fasting.

Whole-blood obtained from the subject is usually processed to obtain either serum or plasma components. As used herein the term “serum” refers to the fraction of whole blood that remains after the suspended materials have been removed. In particular, serum is the fluid that remains after red blood cells, fibrinogen and fibrin have been substantially removed from whole blood. The term “plasma,” as used herein, refers to the fluid fraction of blood as distinguished from the suspended materials. Plasma differs from serum in that plasma contains fibrinogen component that is absent in serum. Plasma is generally prepared with one or more anticoagulants such as, for example, EDTA, citrate, and heparin. As used herein, the terms “plasma” and “serum” connote fluids that are substantially free of red blood cells.

The blood sample (e.g., plasma, serum) may be used directly to prepare a test sample for peptide analysis or may be stored for later use. Conditions for storing blood samples are well known in the art and the methods disclosed herein are suitable for use with blood samples that have been stored in variety of different conditions. For example, the methods are suitable for use with a blood sample that has been stored for a period of time in a liquid state, e.g., at approximately 4° C. to 10° C., or that has been stored for a period of time in a frozen state, e.g., at approximately −20° C. to −80° C. Blood samples that have been frozen are typically thawed at room temperature (e.g., 20° C. to 25° C.) or on ice. And, in any case, following storage, the blood sample is typically mixed (e.g., by vortexing the blood sample) prior to use in the methods.

Aspects of the invention are based on the discovery that diluting the blood sample in water prior to combining the blood sample with other solutions, particularly prior to combining the blood sample with a denaturation buffer, substantially reduces background and improves signal-to-noise ratio in the detection of low molecular weight peptides. Without wishing to be bound by theory it is believed that the improvement is likely due a reduction in the noise and background caused by blood constituents, such as, lipids, high abundance proteins, and other molecules. In the dilution mixture, the ratio of the blood sample to water may be 1:1, 1:2, 1:3, 1:4, 1:5, 1:6, 1:7, 1:8, 1:9, 1:10, or 1:20. However, it should be appreciated that excessive dilution of the blood sample can result in a significant decrease in sensitivity of detection of peptides that are present in blood at low levels, in some instances.

For the analysis of low molecular weight peptides it is often important to separate out larger and more abundant proteins from the blood sample prior to mass spectroscopy. A variety of different separation/enrichment methods are known in the art, including, for example, protein precipitation, affinity purification, ion exchange chromatography, size exclusion chromatography, and ultrafiltration. However, certain limitations associated with these traditional methods can be problematic for downstream analyses. Aspects of the invention are based on the recognition that art known methods for separation and enrichment of low molecular weight peptides can be a significant source of error and variation that impedes development of effective mass spectroscopic based assays, including in the clinical diagnostic setting. It is recognized herein, that for known MALDI-TOF based assays in particular, problematic spot-to-spot and sample-to-sample variation are due, in part, to incomplete and inefficient separation of peptides of interest. Accordingly, aspects of the invention provide improved methods for separation and enrichment of low molecular weight peptides that reduce variation (e.g., spot-to-spot and sample-to-sample variations) and increase sensitivity.

Methods of the invention involve combining a blood sample (serum, plasma) with a denaturation buffer that preferentially denatures and precipitates large, abundant proteins (e.g., albumin, α-macroglobulin, and fibrin) in the blood sample. Thus, according to methods of the invention, when a denaturation buffer is combined with the blood sample, the majority of large, abundant proteins in the blood sample denature and form insoluble aggregates, while most low molecular weight peptides (e.g., up to 50 kDa) in the sample remain in the soluble phase. The soluble phase may then be separated and processed further for peptide analysis. Removal of large, abundant proteins, among other things, improves the signal-to-noise ratio for the detection of smaller peptides by MALDI-TOF mass spectrometry.

As used herein, the term “denaturation buffer” refers to a buffer that effectively denatures large (e.g., greater than 50 kDa), abundant proteins in a blood sample. Denaturation buffers which are suitable for mass spectroscopic methods usually comprise one or more volatile organic acids. As used herein, the term “volatile organic acid” refers to an acid that has at least one COOH group and that readily evaporates at room temperature. A volatile organic acid typically contains 6 or fewer carbon atoms. Non-limiting examples of volatile organic acids include a citric acid, a cyanic acid, a lactic acid, a formic acid, an acetic acid, a propionic acid, and a butyric acid. A volatile organic acid may be trifluoroacetic acid (TFA) or trichloroacetic acid (TCA).

Plasma proteins can be roughly divided into Highly-Abundant Proteins (HAP), Moderately-Abundant Proteins (MAP) and Low-Abundant Proteins (LAP). The HAP fraction may constitute over 96% of the total protein mass and includes proteins such as, for example, Human Albumin (HSA), IgG, Fibrinogen, Transferrin and many other proteins. A reference range for albumin concentrations in blood is 30 to 50 g/L. Albumin has a molecular mass of approximately 67 kDa. Albumin can account for >70% of plasma proteins. In some embodiments, the protein precipitation (crash) that occurs following addition of the denaturation buffer removes a vast majority of proteins over 10 kDa in size and thus over 90% of the plasma proteome. Therefore, in some embodiments, the soluble phase contains peptides <10 kDa and <10% of proteins in plasma (the plasma peptidome/proteome).

In order for the denaturation buffer to effectively denature large, abundant proteins from blood, the denaturation buffer should be capable of overcoming the buffering strength of blood itself. Thus, the concentration of acid in the denaturation buffer may vary depending on the strength of the acid and the manner in which the blood sample is processed prior to combining it with the denaturation buffer. Typically, the concentration of the volatile organic acid after combining with the blood sample is in a range 1% and 50%. Aspects of the invention are based on the discovery that final concentrations of volatile organic acid (e.g., TFA) of greater than 1% when combined the blood sample are effective at denaturing large, abundant proteins. A final concentration of the volatile organic acid of at least 4% when combined the blood sample is particularly effective at denaturing large, abundant proteins.

The denaturation buffer may also comprise an organic solvent to facilitate denaturation and precipitation of large, abundant proteins, and solubilization of low molecular weight proteins. As used herein, the term “organic solvent” refers to a compound containing one or more carbon atoms that dissolves other substances. Non-limiting examples of organic solvents include acetonitrile (ACN), acetone, isopropanol, ethanol, methanol, and combinations thereof. The final concentration of the organic solvent (when combined with the blood sample) may be up to 40% or more. The final concentration of the organic solvent in the combination may 10%, 15%, 20%, 25%, 30%, 40%, 45%, 50%, or more. Once the large, abundant proteins are denatured, the sample is stable and can be stored for future use.

The denaturation buffer may be combined with the blood sample in any appropriate ratio provided that the final concentration of the denaturation buffer is sufficient to precipitate the large, abundant proteins from the blood sample. Typically, the ratio of the denaturation buffer to blood sample is in a range of 1:10 to 10:1. For example, addition of denaturation buffer comprising 8% TFA and 20% ACN to the blood sample (1:1 (buffer:plasma) ratio) results in a final concentration of 4% TFA and 10% ACN and is effective for denaturing and precipitating large, abundant proteins from the blood sample.

After combining the blood sample with the denaturation buffer, the combination is typically incubated for a period of time before the soluble portion is separated from the insoluble protein aggregates. The incubation period should be of a sufficient duration for denaturation and aggregation of the large, abundant proteins to occur, and to enable separation of a soluble fraction enriched in low molecular weight peptides. The combination may be incubated for up to 5 min., 10 min., 15 min., 20 min., 30 min, or more. Typically the combination is incubated at room temperature (e.g., 20° C. to 25° C.). However, incubation may be performed at any appropriate temperature, typically within a range of 4° C. to 37° C.

Following incubation the combination may be spun to pellet the insoluble portion, and isolate the soluble portion (the supernatant). Centrifugation may be performed such that the sample is subjected to centrifugal force of up to 2000 g, 4000 g, 6000 g, 8000 g, 10000 g or more, depending on the duration of the spin, volume of the sample, etc. The combination may be spun for up to 5 min., 10 min., 15 min., 20 min., 30 min., or more. The soluble portion may also be separated from insoluble portion by centrifugation through a filter membrane (e.g., polypropylene PVDF). This approach can be implemented in multi-well plates to increase throughput (e.g., using PVDF filter bottom 96-well microtiter plates and a swinging bucket centrifuge). However, samples can be centrifuged in tubes, or any suitable container. The soluble and insoluble phases can also be separated by filtration devices including ultrafiltration tubes or plates with membranes produced from nitrocellulose, PVDF, glass fiber, composite, etc. As an alternative to centrifugation, the samples can be drawn through filter tubes/plates using a vacuum manifold (negative pressure) or pushed via a pump (positive pressure). If desired, additional centrifugation or filtering steps can be performed to further clarify residual precipitation. Following separation, the soluble portion can be transferred to any type of container, e.g., a container made of glass, polypropylene, polycarbonate, polyvinyl, etc.

### Blood Sample Pretreatment

The blood sample may also be combined with a pre-treatment solution comprising a surfactant to sterilize the blood sample, (free from potential pathogens, hepatitis C, HIV, etc.) and thus, easier and safer to handle. It has been discovered according to some aspects of the invention that combining the blood sample with a pre-treatment solution may increase the signal-to-noise ratio in the detection of certain low molecular weight peptides by mass spectroscopy. These results are unexpected because detergents are generally not considered to be compatible with MALDI or mass spectroscopy because of ion suppression effects. In one embodiment, the use of a pretreatment solution results in a 5-fold increase in signal-to-noise ratio, and a limit of quantitation (LOQ) of 1 nM for a low molecular weight peptide. As used herein, the term “pre-treatment solution” refers to a solution that eliminates and/or inactivates blood pathogens. In particular, pretreatment solutions comprise one or more surfactants. Typically, the surfactant is a non-ionic surfactant. Examples of suitable non-ionic surfactants include: Triton X-45, Triton X-100, Triton X-114 Triton X-165, Triton X-305, Triton X-405, Triton X 705-70, Triton CF10, Tween 20, Tween-80, sodium cholate, β-propriolactone, Octyl glucoside, Decyl maltoside and polyoxyethylene-20 sorbitan monooleate. Others suitable surfactants will be apparent to a skilled artisan.

The pretreatment solution may also include an organophosphate. As used herein, the term “organophosphate” refers to a phosphate containing organic compound. Organophosphates are esters of phosphoric acid and include, for example, alkyl-phosphophates, such as tri-n-butyl phosphate.

Methods of the invention are amenable to high-throughput screening (HTS) implementations. Accordingly, aspects of the invention may be carried out in a multi-well format, for example, a 96-well, 384-well format, or 1,536-well format, and are suitable for automation. In some embodiments, an integrated robot system consisting of one or more robots transports assay microplates between multiple assay stations for blood sample, buffer and/or reagent addition, mixing, incubation, and detection. In some aspects, an HTS system of the invention may prepare, incubate, and analyze many plates simultaneously, further speeding the data-collection process. High throughput implementations for mass spectroscopy and MALDI-TOF are well known in the art. See, for example, Can S A, Annan R S. Curr Protoc Mol. Biol. 2001 May; Chapter 10:Unit 10.21; Somasundaram K, et al., Expert Rev Mol. Diagn. 2009 October; 9(7):695-707; and Ocak S, et al., Proc Am Thorac Soc. 2009 Apr. 15; 6(2):159-70.

### MALDI-TOF Analysis

Matrix-assisted laser desorption/ionization (MALDI) is a soft ionization technique used in mass spectrometry, allowing the analysis of peptides and other biomolecules, which tend to be fragile and fragment when ionized by conventional ionization methods. In MALDI, ionization is triggered by a laser beam (e.g., a nitrogen laser). A matrix is used to protect the peptide from being destroyed by direct laser beam and to facilitate vaporization and ionization. MALDI is typically using with a time-of-flight (TOF) mass spectrometer The TOF measurement procedure is suited to the MALDI ionization process since the pulsed laser takes individual ‘shots’ rather than working in continuous operation. MALDI-TOF instruments are typically equipped with an ion mirror, deflecting ions with an electric field, thereby doubling the ion flight path and increasing the resolution.

A matrix solution is mixed with the sample and spotted onto a MALDI plate (usually a metal plate). Alternatively, the sample can be incubated on matrix pre-spotted surfaces (e.g., Qiagen Turbo MTP chip). In either case, after spotting, solvents in the sample vaporize, leaving only the recrystallized matrix, with peptide distributed throughout the crystals. Typically, the volume of the sample spotted on the MALDI plate is in a range of 1 μL to 10 μL.

The sample can be spotted on the MALDI plate surface using any suitable meanings, e.g., needle, pipette, etc. Samples are allowed sufficient time after spotting on the chip to permit recrystallization of the matrix. On-chip incubations are typically in a range of 1 min. to 25 min. Incubation may be performed at any suitable temperature. Typically, incubation is performed at a temperature in a range of 4° C. to 37° C.

Any suitable matrix molecule may be used in the methods. Suitable matrix molecules are often acidic and act as a proton source to facilitate ionization of the peptide analyte. However, basic molecules may also be used. The matrix molecules often rapidly and efficiently absorb the laser irradiation and may be functionalized with polar groups to facilitate use in aqueous solutions. Non-limiting examples of matrix molecules include 3,5-dimethoxy-4-hydroxycinnamic acid (sinapinic acid or SA), α-cyano-4-hydroxycinnamic acid (alpha-cyano or alpha-matrix, or CHCA or HCCA) or 2,5-dihydroxybenzoic acid (DHB).

Samples are typically analyzed via MALDI-TOF in positive ion reflector mode. Shots are averaged and peptides are quantified by measuring average isotope peak areas relative to that of the internal standards (isotopic peptides) which are present at known concentration. Typically, 500 shots to 2000 shots are averaged. Standard MALDI-TOF parameters such as extraction time, reflector versus linear mode, source and detector voltages, and others can be adjusted, as appropriate, using methods well known in the art.

### Isotopic Peptide Standards

Isotopic peptides are provided that facilitate quantification of peptides using mass spectroscopy by serving as an internal standard. Methods of the invention for determining peptide levels in a subject typically involve detecting peptide in a blood sample from a subject and comparing the detected peptide with an internal standard in order to determine the level of the hepcidin in the blood sample. Detection typically involves measuring the intensity of peaks in a mass spectrum. The mass spectrum is often presented as a vertical bar graph or line plot, in which each bar, or peak, represents an ion having a specific mass-to-charge ratio (m/z) and the length of the bar, or peak, indicates the relative abundance of the ion (peptide). Most of the ions formed in a MALDI mass spectrometer have a single charge, so the m/z value is equivalent to mass itself.

Since a mass spectrometer separates and detects ions of slightly different masses, it readily distinguishes different isotopes of a given element. Taking advantage of this property, methods for determining peptide levels in a subject typically involve adding a known quantity of isotopic peptide to a blood sample obtained from a subject, wherein the isotopic peptide has a molecular weight that differs from the molecular weight of corresponding non-isotopic peptide by a known amount.

Peptides may be synthesized with different quantities of isotopes to produce an isotopic version of a peptide having a known molecular weight shift. Such isotopic peptides can be added at anytime to the blood sample and serve as an internal standard. By comparing peak intensities in the MS spectrum between the non-isotopic target peptide and the isotopic internal standard (which has a known quantity in the sample) the quantity of the target peptide can be determined.

It has been discovered that certain mass shifts can be problematic due to oxidation of the analyte peptide (e.g., methionine oxidation). Thus, mass shifts of 16 Da (and multiples thereof) are typically avoided. It has been discovered that for certain peptides, a mass shift in a range of about 8 Da to 12 Da is desirable. For example, an isotopic hepcidin is provided that has an amino acid sequence as set forth in SEQ ID NO: 3, wherein the arginine at amino acid position 16 is arginine-¹⁵N₄¹³C₆. This isotopic hepcidin has a mass shift of 10 Da compared with the corresponding non-isotopic hepcidin. Compositions and kits comprising the isotopic peptides are also provided.

### Methods for Evaluating and Diagnosing Iron-Associated Disorders

In further aspects of the invention, methods are provided for evaluating iron-associated disorders in a subject based on hepcidin levels. Methods for diagnosing iron-associated disorders in a subject based on hepcidin levels are also provided. As used herein, the term “iron-associated disorder” refers to a disease or condition associated with pathologically high or pathologically low iron levels in the blood of a subject. Alterations in hepcidin levels can lead to pathological changes in blood iron levels, and thus, are linked with iron-associated disorders. Non-limiting examples of iron-associated disorders include, for example, anemia of chronic disease (anemia of inflammation), iron-refractory iron deficiency anemia, rheumatologic disease, inflammatory bowel disease, multiple myeloma, cancer, hereditary hemochromatosis, β-thalassemia, congenital hypoplastic anemia, hemolytic anemia, hereditary sideroachrestic anemia, hereditary sideroblastic anemia, microcytic anemia, macrocytic anemia, megaloblastic anemia, sickle cell anemia and other iron loading anemias.

Methods of the invention involve determining hepcidin levels in a blood sample obtained from a subject. The blood sample is processed according to any of the preparative methods disclosed herein. A sample resulting from the blood processing, which is enriched in low molecular weight peptides, including hepcidin, is subjected to MALDI-TOF mass spectrometry. To facilitate hepcidin quantification, the sample is spiked with an internal standard of isotopic hepcidin at a known concentration. Peaks in the mass spectrum that correspond to isotopic and non-isotopic hepcidin are identified. By comparing intensities of the non-isotopic hepcidin peaks and isotopic hepcidin peaks in the MS spectrum the quantity of the hepcidin in the blood can be determined.

Further methods of the invention involve diagnosing a subject as having the iron-associated disorder, based, at least in part, on hepcidin levels. In some cases, significantly higher hepcidin levels in the subject compared with a normal subject indicate a diagnosis of anemia of chronic disease (anemia of inflammation), iron-refractory iron deficiency anemia, rheumatologic disease, inflammatory bowel disease, multiple myeloma, or cancer. In other cases, significantly lower hepcidin levels in the subject compared with a normal subject indicate a diagnosis of hereditary hemochromatosis, β-thalassemia, congenital hypoplastic anemia, hemolytic anemia, hereditary sideroachrestic anemia, hereditary sideroblastic anemia, microcytic anemia, macrocytic anemia, megaloblastic anemia, sickle cell anemia or another iron loading anemia.

Further methods of the invention involve monitoring hepcidin levels. For example, hepcidin levels can be monitored in a subject (e.g., a human subject) having or suspected of having an iron-associated disorder (e.g., anemia of chronic disease (anemia of inflammation), iron-refractory iron deficiency anemia, rheumatologic disease, inflammatory bowel disease, multiple myeloma, cancer, hereditary hemochromatosis, β-thalassemia, congenital hypoplastic anemia, hemolytic anemia, hereditary sideroachrestic anemia, hereditary sideroblastic anemia, microcytic anemia, macrocytic anemia, megaloblastic anemia, sickle cell anemia and other iron loading anemias). The subject may be an animal model of the iron-associated disorder. The subject may be a participant in, or laboratory subject for, a clinical study (e.g., a study for evaluating the effectiveness of a treatment of an iron-associated disorder and/or the effectiveness of a clinical assay (e.g., prognostic or diagnostic assay)). The subject may be monitored at one or more points in time before, during and/or after receiving a treatment (e.g., an experimental treatment, a clinical treatment, a treatment that is the subject if a clinical investigation) for an iron-associated disorder.

In some embodiments, the methods involve monitoring (e.g., longitudinal monitoring) of the effects of an agent (e.g., a therapeutic candidate, an approved drug) on hepcidin levels in a subject. The monitoring may involve obtaining a baseline hepcidin level (e.g., before treatment) and one or more post-treatment levels in a subject (e.g., animal model). In one example, hepcidin levels are monitored in a subject who is a participant in, or laboratory subject for, a study to evaluate a therapeutic drug (e.g., siRNA, miRNA) that specifically targets hepcidin gene expression. In another example, hepcidin levels are monitored in a subject who is a participant in, or laboratory subject for, a study to evaluate a therapeutic drug (e.g., siRNA, miRNA) that specifically targets expression of a gene (e.g., upstream stimulatory factor 2 (USF2)), the product of which modulates hepcidin levels. In some embodiments, hepcidin levels are monitored in a subject in connection with a treatment that modulates levels of the iron-sensing protein transmembrane protease, serine 6 (TMPRSS6). TMPRSS6 is an upstream serine protease that modulates levels of hepcidin, and germline mutations of TMPRSS6 are associated with Iron Refractory Iron Deficiency Anemia (IRIDA).

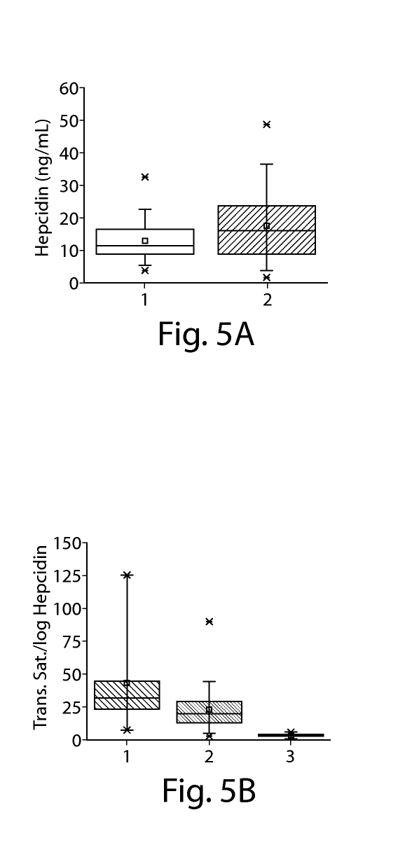

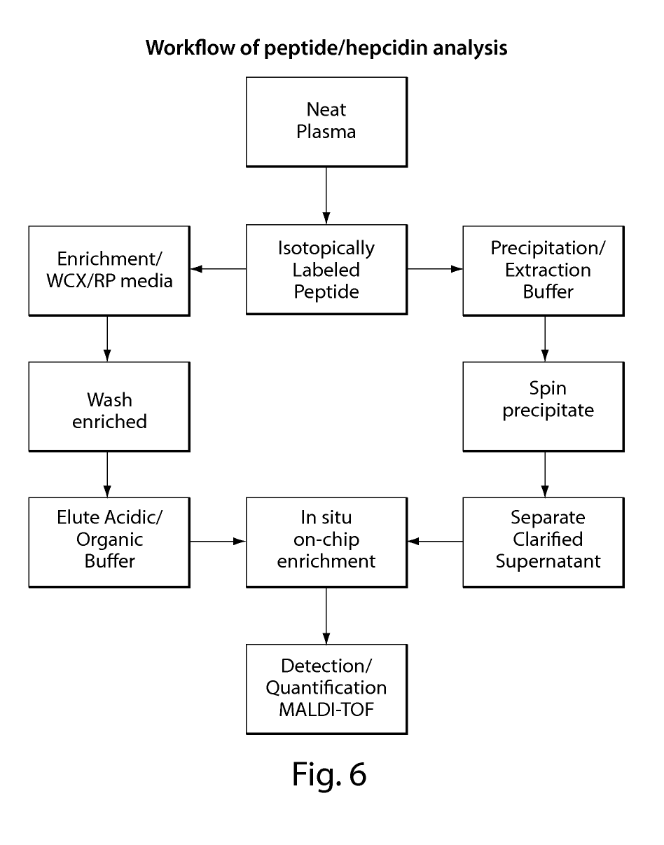

As used herein, the terms “significant difference”, “significantly different”, “significantly higher”, “significantly lower” refer to differences between values that are of a sufficient magnitude to enable reliable identification of a particular effect, e.g., changes in hepcidin levels which are sufficient to inform a diagnosis of an iron-associated disorder. Typically, a significant difference is a difference that is statistically significant according to an appropriate statistical test, e.g., a Student's t-test, an ANOVA, etc. Significant differences between two values may be about 1%, about 5%, about 10%, about 20%, about 30%, about 40%, about 50%, about 60%, about 70%, about 80%, about 90%, about 95%, about 99% of depending on a variety of factors, including, for example, the nature of the value, the number of samples from which a value is derived, the statistical power of a comparison between two values, etc. Significant differences may also be identified between two statistical distributions, e.g., by using an appropriate statistical test to compare distributions (See, e.g., FIG. 5). Similarly, as used herein, values that are “equal” are values that can not reliably be established to be different. Typically, equal values are values between which a statistically significant difference is not found, using an appropriate statistical test, e.g., a Student's t-test, an ANOVA, etc. Thus, equal values may in fact be different, but the differences are not statistically significant.

### Compositions and Kits

Compositions are provided that are useful in any of the preparative methods of the invention. Accordingly, compositions of the invention may comprise any of the following: isotopic peptides, denaturation buffers, pre-treatment solutions, volatile organic acids, organic solvents, organophosphates, surfactants, and matrix molecules, for example.

An exemplary denaturation buffer composition comprises a volatile organic acid and an organic solvent, diluted in water. The concentration of the volatile organic acid and the organic solvent in the composition is sufficiently high such that when combined with a blood sample the desired concentration of the components of the denaturation buffer are present and efficient protein denaturation and precipitation is achieved. For example, a denaturation buffer composition may comprise a volatile organic acid at a concentration in a range of 2% to 16% and an organic solvent at a concentration in a range of 10% to 40%, diluted in water. Such a composition is useful a denaturation buffer when combined with a blood sample at a 1:1 ratio (buffer:blood).

An exemplary pre-treatment solution composition comprises a surfactant and/or a organophosphate. The concentration of the surfactant and organophospate in the composition is sufficiently high such that when combined a blood sample the desired concentration of the components of the pre-treatment solution are present to effect sterilization of the blood sample and/or to improve sensitivity of peptide detection. For example, a pre-treatment solution composition may comprise a surfactant at a concentration in a range of 5% to 10% and/or a organophosphate at a concentration in a range of 5% to 10%. Such a composition is useful as a pre-treatment solution when combined with a blood sample at a ration of 1:9 (solution:blood).

An exemplary isotopic peptide composition comprises an isotopic peptide having a molecular weight in a range of 0.2 kDa to 10 kDa. The isotopic peptide may have a molecular weight in a range of 8 Da to 12 Da greater than the molecular weight of a corresponding non-isotopic peptide. The isotopic peptide may be isotopic hepcidin, which may have an amino acid sequence as set forth in SEQ ID NO: 3, in which the arginine at amino acid position 16 is arginine-¹⁵N₄¹³C₆. Accordingly, a corresponding non-isotopic peptide may be hepcidin having an amino acid sequence as set forth in any one of SEQ ID NO: 1 to 30 or the portion of the sequence of any one of SEQ ID NO: 1, 6, 8, and 10-30 that corresponds to mature hepcidin.

Another exemplary composition comprises a volatile organic acid at a concentration in a range of 1% to 8% and an organic solvent at a concentration in a range of 5% to 20%. The composition may also comprise an organophosphate at a concentration in a range of 0.25% to 2 and a surfactant in a range of 0.25% to 2%. The composition may also comprise a blood sample (e.g., at a concentration of 30% to 60%). The blood sample may be a serum sample, a plasma sample and may have been diluted with water. The composition may comprise an isotopic peptide having a molecular weight in a range of 0.2 kDa to 10 kDa, such as, for example, isotopic hepcidin.

Compositions of the invention may be present in any suitable container. For example, the composition may be in vial, flask, beaker, tube, or a well of a multi-well plate. The container, e.g., well of a multi-well plate, may comprises two chambers separated by a filter membrane, with the composition disposed on the filter membrane in the first chamber. The filter membrane may be polyvinylidene fluoride (PVDF), polyethylene sulfone (PES), polycarbonate, polytetrafluoroethylene (PTFE), or glass fiber, and may have a pore size in a range of 0.2 μm to 1.0 μm.

The composition and agents described herein may be assembled into diagnostic or research kits to facilitate their use in diagnostic or research applications. A kit may include one or more containers housing the components of the invention and instructions for use. Specifically, such kits may include one or more agents described herein, along with instructions describing the intended application and the proper use of these agents. In certain embodiments agents in a kit may be at a concentration suitable for a particular application and for a method of use of the agents. For example, kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of diagnostic products and/or related reagents.

### EXAMPLES

### Example 1

### Development Of A MALDI-TOF Based Assay For Quantification Of Peptide Levels in Blood

A method was developed to detect and quantify target peptides derived from human blood plasma and serum. Although dynamic fluctuations in blood protein content are relatively low compared with urine, blood contains many more proteins covering a large (>10⁹) range of abundance (27, 28). Elements of assay development included determination of the optimal blood background (whole blood, plasma, or serum) and abundant protein depletion and/or selective enrichment procedures. To this end, abundant protein depletion techniques were investigated, including: 1) solvent based protein precipitation using 4-10 fold excess acetone, ethanol, propanol, methanol, and acetonitrile, 2) ultrafiltration and fractionation using low molecular weight cutoff filter tubes, and 3) weak cation exchange enrichment (WCX) of target peptides from diluted and precipitated samples, and a number of alternative techniques. It was discovered that 4-10 fold excess ice-cold acetonitrile caused abundant protein precipitation while permitting subsequent WCX based target peptide enrichment and detection via MALDI-TOF. Several limitations prevented this approach from being useful for subsequent analysis. First, functionalized MALDI chips pre-spotted with MALDI matrix via vacuum sublimation process are not compatible with moderate to high concentrations of acetonitrile (or other organic solvents), and second, the yield of this precipitation and enrichment step was quite low, providing only ≧20 nM sensitivity with ≧200 μL blood plasma input volume.

In further experiments, microscale sample volumes and with minimal upstream treatment of blood plasma prior to analysis were evaluated. Employing the size fractionation and low molecular weight partitioning properties of sublimated matrix spots, the effects of acid extraction coupled with solvent precipitation were evaluated. Addition of 1% TFA to neat blood plasma (sodium heparin treated) resulted in a hazy, waxy sample consistency, and although extremely high endogenous target peptide concentrations could be detected, sensitivity and spot-to-spot precision and reproducibility were very poor. Three parameters were investigated. First, consistent with the microscale approach, conditions for reducing noise and background were sought by testing serial dilutions of untreated blood plasma in water, and compared these with TFA treated and diluted samples. It was discovered that dilution of blood samples enhanced the detection of the low molecular weight peptidome. Without wishing to be bound by theory it is believed that this enhancement is brought about by reducing the noise/background caused by non-peptidic blood constituents (lipids, molecules, and high abundance proteins). Addition of TFA caused limited protein precipitation, and low molecular weight peptide profiles were enhanced by including a centrifugation step directly before sample on-chip incubation.

Through a further series of experiments, it was discovered that higher concentrations of volatile organic acid (>1% TFA final) caused faster, more prolific protein precipitation and was still compatible with maintaining the integrity of the MALDI functionalized chip surface. It appeared that high final concentration of TFA (up to 4%) caused greater and more consistent protein precipitation, such that low end sensitivity of target peptide detection was now achieved and spot-to-spot reproducibility was greatly enhanced. Without wishing to be bound by theory, it is believed that this enhancement was brought about by overcoming the buffering strength of blood plasma with efficient acid extraction of target peptide from other proteins, either carrier molecules or non-specific aggregates, and efficient partitioning into the soluble phase, and by efficient acid-based precipitation of high abundance proteins, e.g. albumin, α-macroglobulin, fibrin, and subsequent partitioning into the insoluble phase. For comparison, addition of 1% TFA final to blood plasma (sodium heparin) resulted in a milky, waxy sample consistency and very minimal apparent target peptide amongst a complex plasma background on the MALDI source. In contrast, addition of 4% TFA final led to a rapid precipitation and the appearance of near 40% total sample volume of precipitate. After a brief centrifugation step, the clarified supernatant gave rise to target peptide quantification down below the ≦20 nM level.

Two further developments enabled a net ≦10 nM detection threshold, a 1:1 dilution of blood plasma with 8% TFA (4% TFA final) thereby effectively removing excess sample and noise from the microscale preparation, and the inclusion of 20% acetonitrile (10% acetonitrile final). The latter development is believed to impact the sample chip, with low concentrations of acetonitrile enhancing the absorptive capacity of the nanostructured prespotted MALDI chip surface. In total, incubation of blood plasma (either sodium heparin or potassium EDTA) with a 1:1 solution of 8% TFA, 20% acetonitrile in water (4% TFA, 10% ACN final), followed by a 15 minute room temperature incubation, gave rise to abundant target peptide extraction and abundant protein precipitation (˜50% total sample volume). A low speed centrifugation step was adequate to allow removal of soluble phase and detection of target peptide to the low nM level, using the stable isotope labeled synthetic target peptide as an internal standard. With this method recovery is ˜100% as the target peptide partitions into the soluble phase; the insoluble phase occupies ˜50% of sample volume post precipitation.

Comparison with Other Methods

Many MS based detection schemes involve an upstream sample preparation and/or fraction/enrichment step in order to foster partitioning of the analyte away from exogenous materials that are ultimately removed by chromatography (reversed phase), precipitation, filtration, or through the use of a solid phase extraction trapping column or SPE plate. In a majority of these methods, in the absence of partitioning/enrichment, the analyte will either not ionize or will be impacted by interfering materials in the analysis and detection phases of the MS (ion suppression).

The effectiveness of a 4% TFA/10% ACN based precipitation, as disclosed herein for blood sample preparation in mass spectroscopy, is unexpected. LCMS (Liquid chromatography tandem mass spectrometry) is not compatible with ≧1% TFA (causes massive ion suppression) nor is it capable of tolerating potential precipitated material which may clog the microbed column or foul the electrospray source. The use of a 4% TFA/10% ACN pretreatment of blood samples prior to WCX enrichment or SELDI-TOF (Surface enhanced laser desorption ionization time-of-flight) analysis would be problematic because the acidification abolishes the cation exchange properties of the peptides in the blood sample rendering the ionizable side chains fully protonated at pH 2. Furthermore, other enrichment methods including metal chelate (Ni NTA) affinity chromatography are not compatible with extreme these acid conditions. As disclosed herein, however, MALDI-TOF is compatible with 4% TFA and its use in the prefractionation/precipitation step facilitates quantitative analysis of low nanomolar endogenous hepcidin in the relatively complex blood samples.

### Sample Pre-Treatment

The above method was used to validate analytical sensitivity and a limit of quantitation (LOQ) of 4 nM (in either sodium heparin or potassium EDTA plasma). In an attempt to define a treatment solution to render blood plasma sterile (free from potential pathogens, hepatitis C, HIV, etc), and thus easier to handle, heat, acoustic/mechanical, and solvent/detergent treatment methods were evaluated. Treatment of neat plasma (prior to 4% TFA/10% ACN denaturation buffer) with 0-1% n-tributyl phosphate (TNBP) and 0-1% Triton X-45 rendered the plasma pathogen-free after 4 hour room temperature incubation (29). The treatment also increased the hepcidin signal intensity and increased detection signal to noise in plasma samples. This result is unexpected because detergents are not typically compatible with MALDI or MS because of ion suppression effects. The net result was better peak resolution of target peptide, a 5-fold increase in signal to noise, and a new LOQ of 1 nM.

### Example 2

### Validation and Clinical Application of a Novel High-Throughput Mass Spectrometry Method for Quantification of Hepcidin in Human Plasma

### Methods

Blood Serum and Plasma Samples

Blood samples were collected from either healthy donors or subjects suspected to have inherited disorders of iron metabolism and their immediate family members. All subjects were recruited and informed consent was obtained under the auspices of a human subjects research protocol. Samples were collected in the early morning and when possible with fasting. Serum and plasma samples were obtained by peripheral venopuncture into vacutainer tubes (Becton Dickinson, Franklin Lakes, N.J.), centrifuged at 2000 g for 10 minutes at 25° C., aliquoted and frozen at −80° C. Plasma assays performed prior to storage included serum Fe/TIBC, ferritin, and sTfR. Erythrocyte studies performed on RBC pellet included zinc protoporphyrin. Pediatric reference samples were obtained as discards from a standard lead screen protocol.

Materials

All solvents were HPLC grade purity (Burdick and Jackson). Ferritin, hemoglobin, bovine serum albumin, and alpha-2 macroglobulin were purchased from Sigma. Filtration discs (0.2 μm) were purchased from Millipore (Billerica, Mass.). Synthetic hepcidin (HepC-25) and stable isotope labeled synthetic HepC-25Arg¹⁵N₄ ¹³C₆ were synthesized by Bachem (Torrance, Calif.). Concentrations of peptide stocks in buffer (0.1% TFA, 5% ACN) were determined in duplicate by amino acid analysis (Molecular Biology Core Facilities at Dana Farber Cancer Institute, Boston, Mass.). All commercial/synthetic peptide solutions were stored at +4° C. under argon. Mass Spec Turbo Chips (384-spot format; 600 μm) and finishing solutions were obtained from QIAGEN (Hilden, Germany). MALDI-TOF experiments were carried out on an Applied Biosystems/MDS SCIEX 4800 instrument.

Plasma Hepcidin Assay

The assay was based on the detection and quantification of endogenous hepcidin relative to that of a stable isotope labeled hepcidin internal standard added to samples at a known concentration. Plasma samples stored at −80° C. were thawed at 25° C. and vortexed briefly. Internal standard (1/20th volume) was mixed with neat plasma and samples were equilibrated for 5 min at 25° C. in 96-well polypropylene microtiter PVDF filter plate wells (Millipore). Pretreatment solution and denaturation buffer were mixed (Final combination—8% TFA/20% ACN/1% TNBP/1% Triton X-45) and added to neat plasma at 1:1. Samples were incubated 15 min on ice. Samples were then subjected to centrifugation 10 min at 3000 g at 25° C. in an Eppendorf 5810 microcentrifuge. In situ hepcidin enrichment was performed by incubating plasma filtrate (5 μL) on the Mass Spec Turbo Chip spot surface for 20 min. under controlled environmental conditions (20° C., 50% relative humidity). Following incubation, plasma was removed and several brief (10 sec) washes (5 μL) were performed using finishing solution prior to MALDI-TOF MS analysis. A standard peptide mixture deposited onto designated spots was used for external chip calibrations. Positive ion reflector mode MS spectra were the average of 1000 shots. Raw data files were exported and analyzed using Data Explorer software (Applied Biosystems v.4.9, Foster City, Calif.). Offline analysis and graphing was performed using Excel (Microsoft Office 2007, Redmond, Wash.) and Origin (OriginLab v.7.0, Springfield, Mass.). Spectra were analyzed manually and relative intensities were determined by obtaining the baseline corrected average of the three initial isotopic peaks for each HepC-25 (2788, 2789, 2790) and HepC-25Arg¹⁵N₄¹³C₆ (2798, 2799, 2800) peak series. HepC-25 concentration was determined from peak area data. The same isotope peaks were selected for analysis throughout experiments to ensure accuracy and consistency. The following equation was used to determine relative HepC-25 concentration from peak area data and known internal standard concentration:

((HepC-25 avg peak area[IS])/IS avg peak area))[HepC-25]

HepC-25 is regular (light) hepcidin peptide and internal standard is the stable isotope labeled internal standard (heavy) synthetic peptide.

Linearity and Analytical Sensitivity

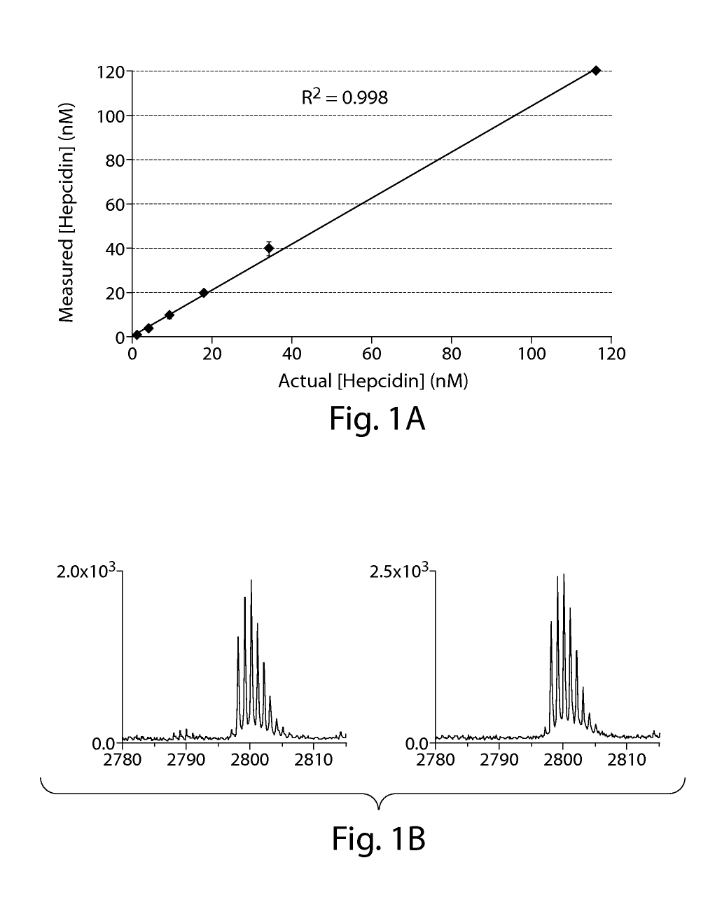

A dilution series of known HepC-25 concentrations was added to aliquots from plasma “blank” samples that exhibited undetectable levels of endogenous hepcidin, as determined by MS analysis (FIG. 1). A single concentration of internal standard was added and multiple replicates were prepared and analyzed. Standard curves were generated based on intensity ratio versus concentration ratio and linear regression was performed. Limit of detection (LOD) was determined as the lowest concentration with signal/noise value of 3, and limit of quantification (LOQ) was defined as lowest concentration with <20% Coefficient of Variation (CV) determined by 5 independent measurements.

Accuracy/Interference

Accuracy (% relative error (RE) ((measured/actual−1)*100)) was determined for samples across the 1-120 nM range (FIG. 1, Table 1). Interference experiments were conducted to measure the effects of plasma components on detection accuracy. Interfering substances (albumin and lipid) and a described hepcidin carrier protein (β-2 macroglobulin) were tested by performing five point dose response experiments in pooled healthy plasma over the range (low, 3/4, 1/2, 3/4, high) from normal (low) to elevated (high) levels (n=3).

Precision

Intra-day and inter-day assay precision was determined by measuring plasma samples representing the low, mid, and high sectors of the linear range of detection (Table 2). In spot-to-spot experiments, mean and standard deviation were determined for duplicate spots. Independent samples prepared in triplicate were used to determine the intra-day reproducibility, while inter-day measurements were taken from independent samples prepared each day over a five day period. The mean and C.V. values for the replicate sets are presented in Table 2.

Preanalytical Experiments

Blood obtained by venopuncture was collected in vacutainer tubes: SST (BD 367986), Na:Heparin (BD 367871), Na:Citrate (BD 369714), K₂:EDTA (BD 367841), K₂:EDTA P800 (BD 366421), K₂:EDTA P100 (BD 801142). Plasma/serum was obtained by centrifugation 2000 g 10 min at room temperature. Platelet poor plasma was obtained by following the first centrifugation step with a second spin at 2500 g for 15 min at room temperature.

Reference Range in Pediatric Normal Sample Set

Plasma from a normal healthy pediatric patient sample set was collected in sodium heparin vacutainer tubes. Whole blood was kept at room temperature for 3 days for lab analysis; plasma was then separated and subjected to short term storage (3-20 days) at −20° C. Plasma assays were performed following HepC analysis, and included Fe, ferritin, and TIBC.

Cross Validation

Cross validation of the described MALDI-TOF method against an existing immunoassay method was performed by independent analysis of plasma samples. Samples were analyzed by immunoassay in 5-10 sample batches over the course of several months. Results were reported in ng/mL units and were kept blinded for the analysis. In MALDITOF experiments, neat plasma samples were mixed with internal standard (1/20th volume) and equilibrated for 5 min at 25° C. in 96-well polypropylene microtiter filter plate wells (Millipore). Binding buffer was added 1:1 and samples were incubated 15 min on ice after which samples were centrifuged for 10 min at 3000 g at 25° C. in an Eppendorf 5810 microcentrifuge. In situ hepcidin enrichment was performed as described above using multi-channel pipette transfers.

Results

Hepcidin was detected in plasma over a wide linear range with LOQ of 1 nM in either sodium heparin or potassium EDTA backgrounds (FIG. 1). Linearity was also examined in serum with a similar observed range and sensitivity. Under the described collection and processing protocols, S/N and resolution of the hepcidin isotopic envelope at the LOQ were (S/N avg. 16.96, Res. avg. 11683.65).

Linearity was good and accuracy was within RE 15% over this range with slightly better error values observed in heparin compared with EDTA (Table 1). Interference experiments measuring dose response from low (normal) to high (elevated) values based on cited reference ranges showed an observable but insignificant (with standard deviations) influence of β-2 macroglobulin on detection (Table 1). Serum albumin showed overall similar results in dose response experiments. Conditions mimicking serial lipidemia were investigated and showed minimal influence at 1%, 0.5%, 0.25% intralipid levels. Additional substances (e.g. billirubin, hemoglobin) showed insignificant interferences.

TABLE 1

| Accuracy/Trueness | | | | | |
|---|---|---|---|---|---|
| Heparin Plasma | | | | | |
| Theoretical (nM) | 4 | 10 | 20 | 40 | 120 |
| Mean (nM) (n = 2) | 4.06 | 9.30 | 17.84 | 34.21 | 116.43 |
| Relative Error (%) | 1.58 | −6.98 | −10.78 | −14.48 | −2.98 |
| EDTA Plasma | | | | | |
| Theoretical (nM) | 4 | 10 | 20 | 40 | 120 |
| Mean (nM) (n = 2) | 4.23 | 8.31 | 17.57 | 33.70 | 104.06 |
| Relative Error (%) | 5.76 | −16.91 | −12.14 | −15.75 | −12.66 |
| Interference | | | | | |
| α-2-macroglobulin | Low | 25% | 50% | 75% | High |
| Heparin Plasma (nM) | 12.65 | 9.58 | 9.78 | 10.01 | 10.00 |
| Standard Deviation (n = 3) | 2.03 | 0.51 | 0.60 | 1.58 | 0.49 |
| EDTA Plasma (nM) | 12.77 | 9.65 | 9.16 | 10.02 | 10.83 |
| Standard Deviation (n = 3) | 3.92 | 1.77 | 1.63 | 1.31 | 1.16 |
| Albumin | Low | 25% | 50% | 75% | High |
| Heparin Plasma (nM) | 11.56 | 18.45 | 9.98 | 10.05 | 10.11 |
| Standard Deviation (n = 3) | 2.56 | 1.87 | 1.32 | 1.24 | 0.96 |
| EDTA Plasma (nM) | 12.87 | 11.01 | 10.56 | 0.92 | 10.07 |
| Standard Deviation (n = 3) | 1.86 | 1.31 | 1.45 | 1.55 | 0.56 |
| Lipid | | 0.25% | 0.50% | 1% | |
| Heparin Plasma (nM) | 12.06 | 11.54 | 10.23 | 10.00 | |
| Standard Deviation (n = 3) | 1.36 | 1.50 | 1.11 | 2.01 | |
| EDTA Plasma (nM) | 12.13 | 10.06 | 10.36 | 9.58 | |
| Standard Deviation (n = 3) | 2.33 | 2.01 | 0.53 | 1.67 | |

TABLE 1.1
Accuracy/Trueness

| Sensitivity | | | | | |
|---|---|---|---|---|---|
| Actual (nM) | 1 | 1 | 1 | 1 | 1 |
| Mean (nM) | 1.09 | 0.87 | 1.11 | 1.07 | 0.97 |
| Relative Error (%) | 9.35 | −12.53 | 11.06 | 6.77 | −3.23 |
| Linear Range | | | | | |
| Actual (nM) | 4 | 10 | 20 | 40 | 120 |
| Mean (nM) (n = 2) | 4.23 | 8.31 | 17.57 | 33.70 | 104.08 |
| Relative Error (%) | 5.76 | −16.91 | −12.14 | −15.75 | −12.66 |

A assessment was performed to a measure of the analytical sensitivity of hepcidin detection method of this example. The assessment involved detecting hepcidin in EDTA-plasma. The assessment involved performing five measurements of independently prepared samples in triplicate, at the lowest concentration at which the % CV (coefficient of variation) and % RE (relative error) are 15% or less. The linear range was determined at no less than five points along the range of detection; % RE did not deviate more than 15%. The results are tabulated in Table 1.1.

In precision measurements, intra-day CV values in both heparin and EDTA plasma followed a general scheme; values were lowest in the mid range and more distinctly higher at the low end (Table 2). Inter-day CV values were noticeably consistent in both heparin (low 14.83, mid 12.08, high 9.73) and EDTA (low 13.05, mid 10.44, high 9.32).

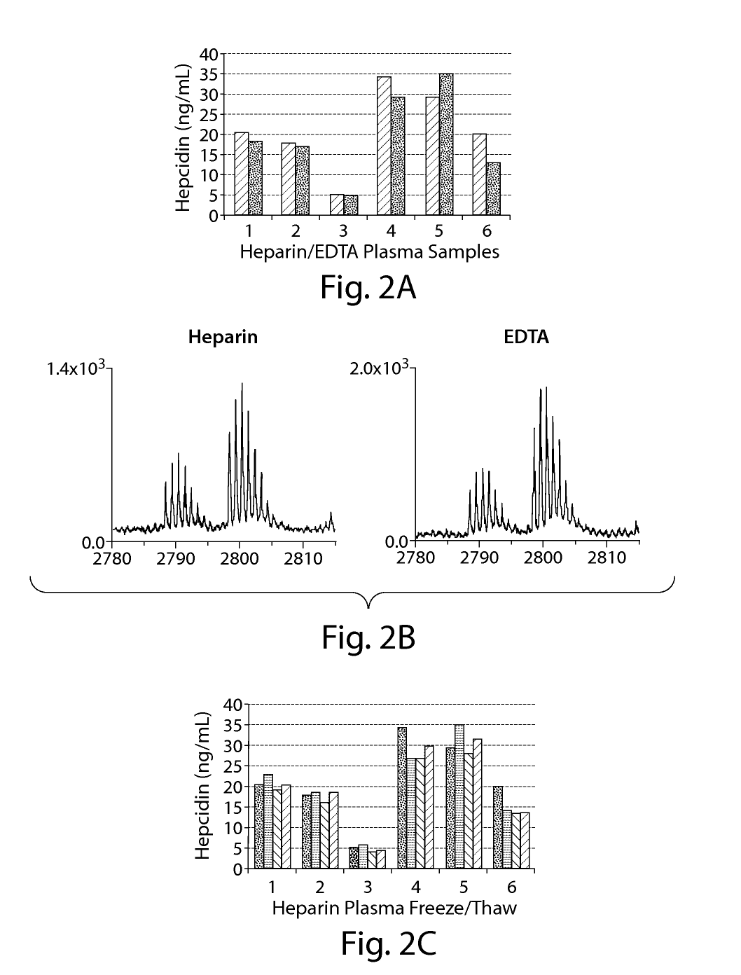

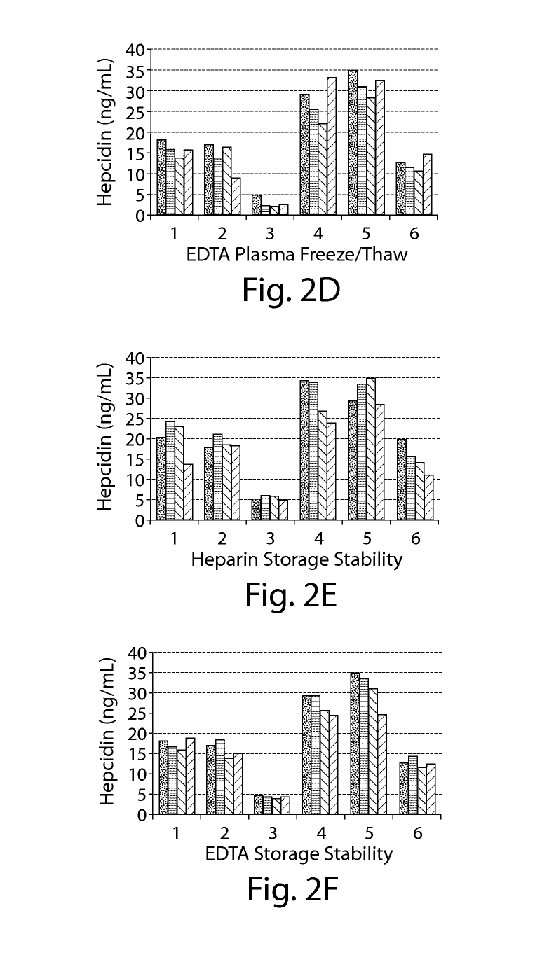

An extensive panel of preanalytical factors was examined to investigate the influences of: successive freeze/thaw treatment, storage duration and temperature, various anticoagulation additives, and plasma processing and platelet depletion methods. Matched heparin and EDTA plasma from the set of anonymous donors was collected (2 females, 4 males, age 23-35, median 27). Endogenous hepcidin levels in these samples ranged from 5-34 ng/mL (21 mean) in heparin and 5-35 ng/mL (20 mean) in EDTA plasma (FIG. 2A). Overall, measurements were similar between plasma types, with consistent HepC m/z peak profile characteristics (FIG. 2B). Successive three day freeze/thaw treatment showed levels of hepcidin remain robust (FIG. 2C-D). Hepcidin levels remained consistent after storage at −80° C. over a 1 day, 1 week, and 1 month period (FIG. 2E-F).

TABLE 2

| | Heparin | | | EDTA | | |
|---|---|---|---|---|---|---|
| | low | mid | high | low | mid | high |
| Intra-day precision | | | | | | |
| Day 1 | | | | | | |
| Mean (nM) (n = 3) | 9.81 | 26.47 | 71.28 | 8.54 | 28.58 | 88.58 |
| CV (%) | 22.15 | 6.86 | 3.60 | 18.88 | 5.68 | 10.20 |
| Day 2 | | | | | | |
| Mean (nM) (n = 3) | 8.61 | 29.97 | 67.13 | 9.83 | 29.62 | 92.10 |
| CV (%) | 15.21 | 12.45 | 2.01 | 24.49 | 6.75 | 7.23 |
| Day 3 | | | | | | |
| Mean (nM) (n = 3) | 11.39 | 24.32 | 90.40 | 10.01 | 30.12 | 80.05 |
| CV (%) | 9.25 | 1.90 | 1.28 | 10.55 | 1.74 | 13.52 |
| Day 4 | | | | | | |
| Mean (nM) (n = 3) | 8.50 | 19.69 | 92.19 | 7.69 | 26.87 | 94.65 |
| CV (%) | 42.11 | 6.50 | 5.69 | 2.25 | 2.24 | 6.12 |
| Day 5 | | | | | | |
| Mean (nM) (n = 3) | 7.96 | 20.70 | 87.57 | 7.62 | 22.98 | 109.45 |
| CV (%) | 16.49 | 5.27 | 3.67 | 24.42 | 1.87 | 1.86 |
| Inter-day precision | | | | | | |
| Mean (nM) (n = 5) | 9.25 | 23.03 | 85.71 | 8.74 | 27.63 | 91.77 |
| CV (%) | 14.63 | 12.09 | 9.73 | 13.05 | 10.44 | 9.32 |

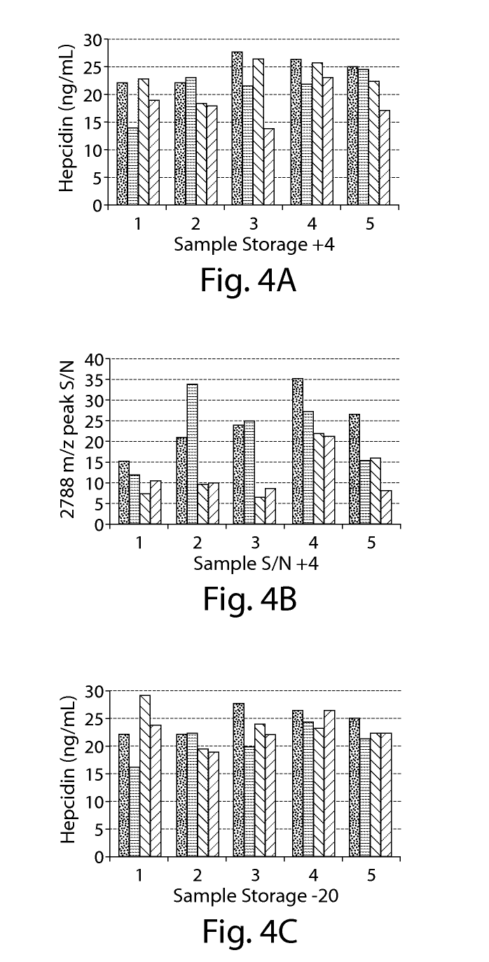

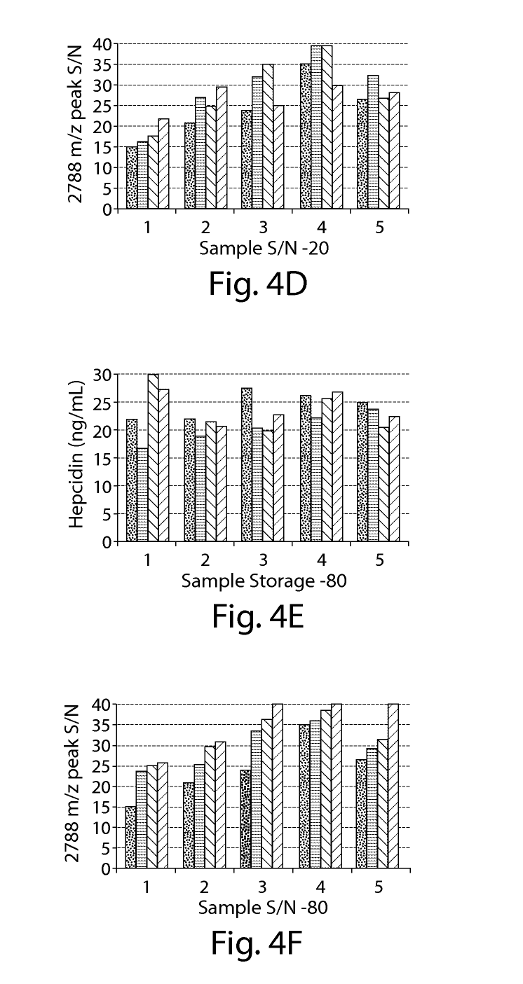

Several pre-storage processing methods were explored to investigate the possible effects of platelet contamination during sample collection and plasma preparation. Normal single-spin RBC collection and plasma removal was compared to platelet depletion methods including: plasma filtration through either 0.2 μm PVDF or polycarbonate filter tubes and a 2-step differential centrifugation procedure. The filtration methods provided distinct results with regard to HepC S/N and replicate sample measurement precision. The 2-step spin method provided a distinct enhancement in S/N and better resistance to long term storage compared with normal single-spin control samples (FIG. 2 and FIG. 4).

To investigate the influence of anti-coagulation tube additives, blood was collected in sodium citrate, sodium heparin, potassium EDTA and EDTA tubes supplemented with plasma protein stabilizing agents (P800 and P100). These samples were subjected to the 2-step spin “platelet depletion” method and monitored over time (1 day, 1 week, and 1 month) and after multi-temperature (+4° C., −20° C., and −80° C.) storage (FIG. 4). Measured hepcidin levels were comparable between plasma types, while storage at +4° C. resulted in a significant decrease in HepC and signal/noise (S/N) after 1 week and beyond. In contrast, S/N of the samples at −20 remained high and at −80° C. even increased over time. The plasma protein stabilizer tubes contributed to improved measurement precision both over time and under various storage temperatures (FIG. 4A-C). Sodium heparin samples showed variability in measurements and overall lower S/N, while EDTA, P800, and P100 samples exhibited increased measurement precision and high S/N values over time (FIG. 4D-F).

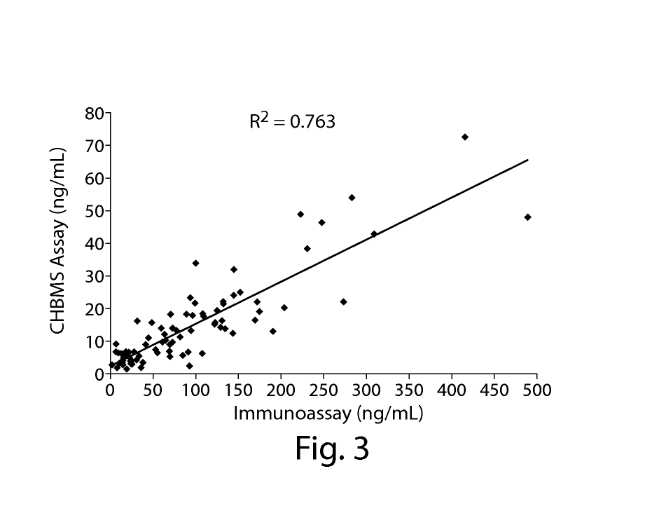

The described plasma hepcidin assay was subjected to cross-validation against a previously described plasma immunoassay. A single set of samples previously analyzed by immunoassay and archived at −80° C. were thawed at room temperature. Samples were prepared using a modification to 96-well plate format and spotted in batch for MALDI-TOF. The data from this set (FIG. 3) show a clear correlation between the CHBMS hepcidin assay and the immunoassay (R2 =0.763; n=87). An offset in hepcidin numbers is observed when comparing plasma data collected by the two methods (FIG. 3). The observed offset may result from differences in the accuracy and/or precision of the methods.

### Analysis of Iron Refractory Iron Deficiency Anemia (IRIDA) Samples

Sample sets of plasma samples from Iron Refractory Iron Deficiency Anemia (IRIDA) patients and their family members were collected and analyzed. Plasma samples were thawed at room temperature and analyzed using the 96-well format and analyzed in a single batch MALDI-TOF run. Hepcidin results demonstrated no significant difference (P=0.025) between homozygous null (+/+) patients and heterozygous affected (+/−) patients. There was a significant difference (P=0.006) between homozygous null (+/+) and homozygous affected (−/−) patients. Use of the iron index (transferrin % saturation/log₁₀ hepcidin) enabled the differentiation between homozygous null (+/+) patients (FIG. 5B, box 1) and heterozygous affected (+/−) (P=0.006) patients (FIG. 5B, box 2).

IRIDA is a disease characterized by patients with iron deficiency anemia unresponsive to oral iron therapy. In addition, these patients have congenital hypochromic, microcytic anemia, or a genetically-based iron-restricted small volume red blood cell phenotype, low transferrin saturation, and no hematological improvement following treatment with oral iron; limited response to parenteral treatment.

IRIDA is linked to germline mutations in TMPRSS6, a type II transmembrane serine protease produced in the liver that regulates the expression of hepcidin. Use of the transferrin sat/log₁₀ hepcidin index (above) enables the detection of heterozygous TMPRSS6 mutations—patients of which may be at risk for iron deficiency in infancy. These results indicate that diagnosis using the hepcidin-based index is possible and may be used for individuals at greatest risk for iron deficiency (e.g., pediatric individuals). The results also indicate that the hepcidin-based index may be used for evaluating and recommending treatments, e.g., treatments other than oral iron therapy, in the prevention of cognitive and developmental deficiencies.

### Example 3

### A Method Outline For MALDI-TOF Based Detection Of Hepcidin

Hepcidin can be detected and quantified in blood PLASMA AND SERUM:
Plasma collection tubes may be used that contain anti-coagulation reagents, sodium citrate, sodium heparin, potassium EDTA, and potassium EDTA tubes with protease inhibitor cocktails.
Serum separation tubes (SST) containing agar gel may be used.
   Hepcidin can be quantified using many types of Internal Standards:
A useful standard is a stable isotope labeled synthetic hepcidin peptide which can include ¹³Cor¹⁵N labels on any amino acid in the hepcidin sequence in any combination (¹³C or¹⁵N single or double labels).
A useful standard is a stable isotope labeled synthetic hepcidin peptide has the ¹³C/¹⁵N double label on Arg16 giving it a mass difference of +10 Da compared with unlabeled synthetic hepcidin—or any other combination the gives a mass difference of +10 Da.
Alternative standards include any natural or synthetic peptide, synthetic peptides similar to hepcidin (i.e., HepC-24 (missing a single amino acid) or other permutations or modifications.
Internal standard can be added to plasma at any time prior to analysis and incubation can be at +4° C. to +25° C., although addition to neat plasma and equilibration for 5 min. room temperature prior to subsequent reagent addition may be preferred in some instances.
   Solvent/Detergent treatment (TNBP/Triton X-45 0-1% final) prior to denaturation/protein precipitation enhances the hepcidin signal intensity and signal/noise significantly.
Treatment may involve addition of mixture of TNBP/Triton X-45 in water 1/20^{th} volume to neat plasma following the internal standard addition/incubation
Final concentrations of TNBP/Triton X-45 in range of 0-1%, e.g., 0.5%.
Incubate 0-4 hours at +4° C. to +37° C.
May be carried out at pH 4.5 to pH 8.4.
May be used with any plasma/serum type.
May be used with any internal standard type.
May substitute Triton X-45 with any of the Triton series of surfactants, sodium cholate, Tween-80, β-propriolactone, polyoxyethylene-20 sorbitan monooleate
May treat with or without TNBP.
   After solvent/detergent treatment, plasma denaturation buffer is added and mixed:
Add 8% TFA/20% ACN in water in a 1:1 ratio with blood to achieve a final concentration of 4% TFA/10% CAN.
Incubation at room temperature for 15 minutes.
The ratio of buffer:plasma can range from 1:10 to 10:1
TFA concentration can range 1% to 50% final
ACN concentration can range 0% to 40% final
Incubation can range 0 minutes to indefinite at +4° C. to +37° C.
Once denatured, the plasma/hepcidin is very stable and can be stored for future use.
8% (4% final) citric acid or formic acid (with 10% final ACN) may alternatively be used
Acetone, ethanol, methanol, or isopropanol can be substituted for acetonitrile (and combined with 4% TFA).
   Separation of denatured plasma soluble hepcidin-containing phase:
Following precipitation, samples are centrifuged and the clarified soluble phase is removed and transferred to fresh vessels for analysis.
Centrifugation may be performed using PVDF filter bottom 96-well microtiter plates in a swinging bucket centrifuge, 2000×g 10-20 minutes.
Samples can be centrifuged in tubes, 96-well microtiter plates, or any type of container;
The soluble supernatant can be transferred to any type of container, e.g., a container made of glass, polypropylene, polycarbonate, polyvinyl, etc.
The soluble and insoluble phases can be separated by filtration devices including ultrafiltration tubes or plates with nitrocellulose, PVDF, glass fiber, composite, etc. membranes.
In place of centrifugation, the samples can be drawn through filter tubes/plates using a vacuum manifold (negative pressure) or pushed via a pump (positive pressure).
   The clarified extracts can be stored or spotted directly on the MALDI chip:
The extracts may be overlayed with argon gas, seal the tubes/plates prior to storage at +4° C. to +37° C. or transport and spotting on the MALDI chip surface.
Spots (5 μL) are incubated on 600 μm vacuum sublimated matrix pre-spotted MALDI chips (e.g., Qiagen Turbo MTP), fixed with CHCA matrix.
Spots are incubated for 20 minutes at +25° C. at 50% relative humidity.
Spots are washed 5 times with 5 μL ammonium phosphate buffer (e.g., Qiagen finishing solution).

### Alternatives:

If desired, a quick spin 2000 g for 2 minutes at +4° C. to +25° C. can be performed to further clarify residual precipitation.
Sample sizes can range (1 μL to 10 μL) and can be transferred to the MALDI plate surface using a needle, pipette, etc. and either mixed with MALDI matrix and spotted on stainless steel or incubated on matrix pre-spotted surface (i.e., Qiagen Turbo MTP chip).
CHCA matrix is typically used, but alternatives, including DHB and Sinapinic Acid can be used.
On-chip incubation can be 1-25 minutes +4° C. to +37° C.
Post on-chip incubation samples may be removed by pipette and spots may be subjected to 3-5 washes 10 seconds each with 10-20 mM ammonium phosphate, sodium phosphate, potassium phosphate, with/without 0-10% ACN. Water and a number of dilute buffers may also be used.

### MALDI-TOF Analysis:

Sample are analyzed via MALDI-TOF in positive ion reflector mode; 1000-2000 shots are averaged and hepcidin is quantified by measuring average isotope peak area of hepcidin relative to that of the internal standard which is present at known concentration.
MALDI-TOF settings:
   1000 shots 10 subspectra of 10 shots each; delayed extraction 400 ns; positive ion reflector mode.
Alternatives
   500-2000 shots; delayed extraction 200-600 ns; reflector or linear mode, varying source and detector voltages, etc.

### Example 4

### Enrichment and Detection of Hepcidin from Different Plasma Sources

### Detection and Quantification of Hepcidin in Cynomolgus Monkey Plasma and Sera Sample Enrichment

Isotopic peptides are provided that facilitate quantification of peptides using mass spectroscopy by serving as an internal standard. The isotopic peptides are added to neat plasma or serum samples at a known concentration.
Neat plasma or serum samples from the monkey (and containing the isotopic peptides) are subjected to chromatographic separation using a weak cation exchange resin (silica and/or agarose).
The resin is washed with a low salt aqueous buffer (0.1-1% KCl)
The sample is eluted from the resin with an acidic aqueous buffer (0.5-5% TFA with 0.1-10% ACN).

### Detection and Quantification of Hepcidin in Mouse Plasma and Sera

### Sample Enrichment

Isotopic peptides are provided that facilitate quantification of peptides using mass spectroscopy by serving as an internal standard. The isotopic peptides are added to neat plasma or serum samples at a known concentration.
Neat plasma or serum samples from the mouse (and containing the isotopic peptides) are subjected to chromatographic separation using reverse phase resin (silica and/or agarose).
The resin is washed with a mild organic buffer (0.1-1.0% ACN).
The sample is eluted from the resin with a high organic buffer (50-70% ACN).
The eluate is subjected to conditions that promote evaporation of the ACN to an organic ACN content of <40% or to complete dryness.
The eluate is then reconstituted in an aqueous buffer (0.1-1% TFA, 0.1-5% ACN). Several fold concentrations can be made through this technique

### MALDI-TOF Preparation:

The eluate from either of the above Sample Enrichment methods can be stored or spotted directly on the MALDI chip:
The eluates may be overlayed with argon gas and sealed in tubes prior to storage at +4° C. to +37° C. or transport and spotting on the MALDI chip surface.
Spots (5 μL) are incubated on 600 μm vacuum sublimated matrix pre-spotted MALDI chips (e.g., Qiagen Turbo MTP), fixed with CHCA matrix.
Spots are incubated for 20 minutes at +25° C. at 50% relative humidity.
Spots are washed 5 times with 5 μL ammonium phosphate buffer (e.g., Qiagen finishing solution).
1. Rifai N, Gillette M A, Can S A. Protein biomarker discovery and validation: the long and uncertain path to clinical utility. Nat Biotechnol 2006; 24:971-83.
2. Diamandis E P, Hanash S, Lopez M, Can S, Petricoin E F, 3rd. Protein quantification by mass spectrometry: is it ready for prime time? Clin Chem 2009; 55:1427-30.
3. Addona T A, Abbatiello S E, Schilling B, Skates S J, Mani D R, Bunk D M, et al. Multi-site assessment of the precision and reproducibility of multiple reaction monitoring-based measurements of proteins in plasma. Nat Biotechnol 2009; 27:633-41.
4. Can S A, Annan R S. Overview of peptide and protein analysis by mass spectrometry. Curr Protoc Mol Biol 2001; Chapter 10:Unit 10 21.
5. Krause A, Neitz S, Magert H J, Schulz A, Forssmann W G, Schulz-Knappe P, Adermann K. LEAP-1, a novel highly disulfide-bonded human peptide, exhibits antimicrobial activity. FEBS Lett 2000; 480:147-50.
6. Park C H, Valore E V, Waring A J, Ganz T. Hepcidin, a urinary antimicrobial peptide synthesized in the liver. J Biol Chem 2001; 276:7806-10.
7. Nemeth E, Tuttle M S, Powelson J, Vaughn M B, Donovan A, Ward D M, et al. Hepcidin regulates cellular iron efflux by binding to ferroportin and inducing its internalization. Science 2004; 306:2090-3.
8. De Domenico I, Ward D M, Langelier C, Vaughn M B, Nemeth E, Sundquist W I, et al. The molecular mechanism of hepcidin-mediated ferroportin down-regulation. Mol Biol Cell 2007; 18:2569-78.
9. Nicolas G, Bennoun M, Porteu A, Mativet S, Beaumont C, Grandchamp B, et al. Severe iron deficiency anemia in transgenic mice expressing liver hepcidin. Proc Natl Acad Sci USA 2002; 99:4596-601.
10. Roy C N, Mak H H, Akpan I, Losyev G, Zurakowski D, Andrews N C. Hepcidin antimicrobial peptide transgenic mice exhibit features of the anemia of inflammation. Blood 2007; 109:4038-44.
11. Du X, She E, Gelbart T, Truksa J, Lee P, Xia Y, et al. The Serine Protease TMPRSS6 Is Required to Sense Iron Deficiency. Science 2008.
12. Finberg K E, Heeney M M, Campagna D R, Aydinok Y, Pearson H A, Hartman K R, et al. Mutations in TMPRSS6 cause iron-refractory iron deficiency anemia (IRIDA). Nat Genet. 2008; 40:569-71.
13. Melis M A, Cau M, Congiu R, Sole G, Barella S, Cao A, et al. A mutation in the TMPRSS6 gene, encoding a transmembrane serine protease that suppresses hepcidin production, in familial iron deficiency anemia refractory to oral iron. Haematologica 2008; 93:1473-9.
14. Roetto A, Papanikolaou G, Politou M, Alberti F, Girelli D, Christakis J, et al. Mutant antimicrobial peptide hepcidin is associated with severe juvenile hemochromatosis. Nat Genet. 2003; 33:21-2.
15. Pietrangelo A. Hereditary hemochromatosis. Biochim Biophys Acta 2006; 1763:700-10.
16. Nemeth E, Ganz T. Hepcidin and iron-loading anemias. Haematologica 2006; 91:727-32.
17. Papanikolaou G, Tzilianos M, Christakis J I, Bogdanos D, Tsimirika K, Macfarlane J, et al. Hepcidin in iron overload disorders. Blood 2005.
18. Semrin G, Fishman D S, Bousvaros A, Zholudev A, Saunders A C, Correia C E, et al. Impaired intestinal iron absorption in Crohn's disease correlates with disease activity and markers of inflammation. Inflamm Bowel Dis 2006; 12:1101-6.
19. Detivaud L, Nemeth E, Boudjema K, Turlin B, Troadec M B, Leroyer P, et al. Hepcidin levels in humans are correlated with hepatic iron stores, hemoglobin levels, and hepatic function. Blood 2005; 106:746-8.
20. Harrison-Findik D D, Schafer D, Klein E, Timchenko N A, Kulaksiz H, Clemens D, et al. Alcohol metabolism-mediated oxidative stress down-regulates hepcidin transcription and leads to increased duodenal iron transporter expression. J Biol Chem 2006; 281:22974-82.
21. Nemeth E, Valore E V, Territo M, Schiller G, Lichtenstein A, Ganz T. Hepcidin, a putative mediator of anemia of inflammation, is a type II acute-phase protein. Blood 2003; 101:2461-3.
22. Ganz T, Olbina G, Girelli D, Nemeth E, Westerman M Immunoassay for human serum hepcidin. Blood 2008; 112:4292-7.
23. Nemeth E, Rivera S, Gabayan V, Keller C, Taudorf S, Pedersen B K, Ganz T. IL-6 mediates hypoferremia of inflammation by inducing the synthesis of the iron regulatory hormone hepcidin. J Clin Invest 2004; 113:1271-6.
24. Jaskolla T W, Karas M, Roth U, Steinert K, Menzel C, Reihs K. Comparison Between Vacuum Sublimed Matrices and Conventional Dried Droplet Preparation in MALDI-TOF Mass Spectrometry. J Am Soc Mass Spectrom 2009.
25. Gerber S A, Rush J, Stemman O, Kirschner M W, Gygi S P. Absolute quantification of proteins and phosphoproteins from cell lysates by tandem MS. Proc Natl Acad Sci USA 2003; 100:6940-5.
26. Poetsch A, Schlusener D, Florizone C, Eltis L, Menzel C, Rogner M, et al. Improved identification of membrane proteins by MALDI-TOF MS/MS using vacuum sublimated matrix spots on an ultraphobic chip surface. J Biomol Tech 2008; 19:129-38.
27. Corthals G L, Wasinger V C, Hochstrasser D F, Sanchez J C. The dynamic range of protein expression: a challenge for proteomic research. Electrophoresis 2000; 21:1104-15.
28. Anderson N L, Anderson N G. The human plasma proteome: history, character, and diagnostic prospects. Mol Cell Proteomics 2002; 1:845-67.
29. Piet M P, Chin S, Prince A M, Brotman B, Cundell A M, Horowitz B. The use of tri(n-butyl)phosphate detergent mixtures to inactivate hepatitis viruses and human immunodeficiency virus in plasma and plasma's subsequent fractionation. Transfusion 1990; 30:591-8.

AMINO ACID SEQUENCES

>gi|10863973|ref|NP_066998.1| hepcidin preproprotein [Homo sapiens]
SEQ ID NO: 1
MALSSQIWAACLLLLLLLASLTSGSVFPQQTGQLAELQPQDRAGARASWMPMFQRRRRRDTHFPICIFCCGCCHRSK
CGMCCKT
>gi|10863973|ref|NP_066998.1| hepcidin proprotein [Homo sapiens]
SEQ ID NO: 2
VFPQQTGQLAELQPQDRAGARASWMPMFQRRRRRDTHFPICIFCCGCCHRSKCGMCCKT
>gi|10863973|ref|NP_066998.1| hepcidin-25 [Homo sapiens]
SEQ ID NO: 3
DTHFPICIFCCGCCHRSKCGMCCKT
>gi|10863973|ref|NP_066998.1| hepcidin-22 [Homo sapiens]
SEQ ID NO: 4
FPICIFCCGCCHRSKCGMCCKT
>gi|10863973|ref|NP_066998.1| hepcidin-20 [Homo sapiens]
SEQ ID NO: 5
ICIFCCGCCHRSKCGMCCKT
>gi|14211542|ref|NP_115930.1| hepcidin precursor [Mus musculus]
SEQ ID NO: 6
MALSTRTQAACLLLLLLASLSSTTYLHQQMRQTTELQPLHGEESRADIAIPMQKRRKRDTNFPICIFCCK
CCNNSQCGICCKT
>gi|14211542|ref|NP_115930.1| Hepcidin-25 [Mus musculus]
SEQ ID NO: 7
DTNFPICIFCCKCCNNSQCGICCKT
>gi|156530243|gb|ABU75218.1| hepcidin preproprotein [Macaca fascicularis]
SEQ ID NO: 8
MALSSQIWATCLLLLLLLASLTSGSVFPQQTGQLAELQPQDRAGARASWTPMLQRRRRRDTHFPICIFCC
GCCHRSKCGMCCR
>gi|156530243|gb|ABU75218.1| Hepcidin-25 [Macaca fascicularis]
SEQ ID NO: 9
DTHFPICIFCCGCCHRSKCGMCCR
>gi|109124350|ref|XP_001094273.1| PREDICTED: hepcidin isoform 1 [Macaca
mulatta]
SEQ ID NO: 10
MALSSQIWATCLLLLLLLASLTSGSVFPQQTGQLAELQPQDRAGARASWTPMLQRRRRRDTHFPICIFCC
GCCHRSKCGMCCRT
>gi|157841284|ref|NP_001103163.1| hepcidin [Pan troglodytes]
SEQ ID NO: 11
MALSSQIWAACLLLLLLLASLTSGSVFPQQTGQLAELQPQDRAGARASWMPMLQRRRRRDTHFPICIFCC
GCCHRSKCGMCCKT
>gi|197097356|ref|NP_001127676.1| hepcidin precursor [Pongo abelii]
SEQ ID NO: 12
MALSSQIWAACLLLLLLLASLTSGSVFPQQTGQLAELQPQDRAGARAGWTPMLQRRRRRDTHFPIYIFCC
GCCHRSKCGMCCKT
>gi|332262028|ref|XP_003280066.1| PREDICTED: hepcidin-like isoform 1
[Nomascus leucogenys]
SEQ ID NO: 13
MALSSQIWAACLLLLLLLASLTSGSVFPQQTGQLAELQGQLDRAGARAGWTPMLQRRRRRDTHFPICIFC
CGCCHRSKCGMCCKT
>gi|332262030|ref|XP_003280067.1| PREDICTED: hepcidin-like isoform 2
[Nomascus leucogenys]
SEQ ID NO: 14
MALSSQIWAACLLLLLLLASLTSGSVFPQQTGQLAELQGQLDRAGARAGWTPMLQRRRRRDTHFPICIFC
CGCCHRSKCGMCCKT
>gi|296233549|ref|XP_002762058.1| PREDICTED: hepcidin-like [Callithrix
jacchus]
SEQ ID NO: 15
MALSSQIWAVCLFLLLLLASLTSGFVFPQQAGQLTELQPQDRAGARASWMPMIQRRRRRDTHFPICIFCC
GCCRQSNCGMCCKT
>gi|301771027|ref|XP_002920925.1| PREDICTED: hepcidin-like [Ailuropoda
melanoleuca]
SEQ ID NO: 16
MALSTRIQAACLLSLLLASLASGSVFPHQTGQLAALQAQDAAGAEAGLMPALPRLRRRDTHFPICLFCCG
CCNKSKCGICCKT
>gi|167583522|ref|NP_001107980.1| hepcidin [Bos taurus]
SEQ ID NO: 17
MALNTQIRATCLLLLVLLSLTSGSVLPPQTRQLTDLQTKDTAGAAAGLTPVLQRRRRDTHFPICIFCCGC
CRKGTCGMCCRT
>gi|305855198|ref|NP_001182241.1| hepcidin [Ovis aries]
SEQ ID NO: 18
MALSTQTRATCLLLLVLLSLTSGSVLPPQTRQLTDLQTQHTAGAAAGLTPVLQRRRRDTHFPICIFCCGC
CRKGTCGICCKT
>gi|47523042|ref|NP_999282.1| hepcidin precursor [Sus scrofa]
SEQ ID NO: 19
MALSVQIRAACLLLLLLVSLTAGSVLPSQTRQLTDLRTQDTAGATAGLTPVAQRLRRDTHFPICIFCCGC
CRKAICGMCCKT
>gi|55742661|ref|NP_001007141.1| hepcidin precursor [Canis lupus familiaris]
SEQ ID NO: 20
MALSTRIQAACLLLLLLASVASVSVLPHQTGQLTDLRAQDTAGAEAGLQPTLQLRRLRRRDTHFPICIFC
CGCCKTPKCGLCCKT
>gi|270047475|ref|NP_001161799.1| hepcidin [Equus caballus]
SEQ ID NO: 21
MALNTNIRAACLLLLLLASLTSGSVLPHQTRQLADLQTQDAAGMAGAAAGLMPGLHQLRRRDTHFPICTL
CCGCCNKQKCGWCCKT
>gi|16758218|ref|NP_445921.1| hepcidin precursor [Rattus norvegicus
SEQ ID NO: 22
MALSTRIQAACLLLLLLASLSSGAYLRQQTRQTTALQPWHGAESKTDDSALLMLKRRKRDINFPICLFCC
KCCKNSSCGLCCIT
>gi|34304034|ref|NP_899080.1| hepcidin-2 [Mus musculus]
SEQ ID NO: 23
MMALSTRTQAACLLLLLLASLSSTTYLQQQMRQTTELQPLHGEESRADIAIPMQKRRKRDINFPICRFCC
QCCNKPSCGICCEE
>gi|147901496|ref|NP_001090729.1| hepcidin 1 [Xenopus (Silurana) tropicalis]
SEQ ID NO: 24
MKPVPICCLLLLLSFICHRGHSASLSGNEVTVTGNQIPETQMEESNALEPLLRSKRQSHLSICVHCCNCC
KYKGCGKCCLT
>gi|318096761|ref|NP_001187130.1| hepcidin [Ictalurus punctatus]
SEQ ID NO: 25
MRAMSIACAVAVIIACVCALQSAALPSEVRLDPEVRLEEPEDSEAARSIDQGVAAALAKETSPEVRFRTK
RQSHLSLCRYCCNCCKNKGCGFCCRF
>gi|45387595|ref|NP_991146.1| hepcidin-1 precursor [Danio rerio]
SEQ ID NO: 26
MKLSNVFLAAVVILTCVCVFQITAVPFIQQVQDEHHVESEELQENQHLTEAEHRLTDPLVLFRTKRQSHL
SLCRFCCKCCRNKGCGYCCKF
>gi|66571313|ref|NP_001018873.1| hepcidin-2 precursor [Danio rerio]
SEQ ID NO: 27
MKLSNVFLAAVVILTCVCVFQITAVPFIQQVQDEHHVESEELQENQHLTEAEHRLADPLVLFRTKRQSHL
SLCRFCCKCCRNKGCGYCCKF
>gi|319412238|ref|NP_001188323.1| hepcidin [Ictalurus punctatus]
SEQ ID NO: 28
MRPMSIACAVAVIIACVCALQSAALPSEVRLDPEVRLEEPEDSEAARSIDQGVAAALAKETSPEVLFRIK
RQSHLSLCRYCCNCCKNKGCGFCCRF
>gi|148230158|ref|NP_001090730.1| hepcidin antimicrobial peptide 2 [Xenopus
(Silurana) tropicalis]
SEQ ID NO: 29
MKSLLLCCLLLLLSLICHRGHSASLSGNEIKAPEHPISESEQGESDALGPLFRTKRHLNICVYCCKCCKK
QKGCGMCCFT
>gi|213512933|ref|NP_001134321.1| Hepcidin-1 [Salmo salar]
SEQ ID NO: 30
MKAFSVAVAVVVVLACMFILESTAVPFSEVRTEEVESIDSPVGEHQQPGGTSMNLPMHFRFKRQSHLSLC
RWCCNCCHNKGCGFCCKF

This invention is not limited in its application to the details of construction and the arrangement of components set forth in this description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A method for detecting a peptide in a subject, the method comprising:
(a) processing a blood sample obtained from a subject by:
(i.) combining the blood sample with a denaturation buffer comprising a volatile organic acid, wherein the concentration of the volatile organic acid in the combination is between 1% and 50%; and
(ii.) separating a soluble fraction from an insoluble fraction of the combination; and
(b) performing MALDI-TOF mass spectrometry on the soluble fraction to detect the peptide.

2. The method of claim 1, wherein the concentration of the volatile organic acid in the combination is 1%.

3. The method of claim 1, wherein the concentration of the volatile organic acid in the combination is 4%.

4. (canceled)

5. The method of claim 1, wherein the volatile organic acid is trifluoroacetic acid (TFA).

6. The method of claim 1, wherein the denaturation buffer further comprises an organic solvent.

7-10. (canceled)

11. The method of claim 1, wherein processing the test sample further comprises adding a pre-treatment solution comprising a surfactant to the blood sample prior to step (i.).

12-17. (canceled)

18. The method of claim 1, wherein the peptide has a molecular weight in a range of 0.3 kDa to 10 kDa.

19. The method of claim 1, wherein the peptide is hepcidin.

20-26. (canceled)

27. A method for detecting a peptide in a plurality of subjects, the method comprising:
implementing the method of claim 1 in a multiplex format to detect the peptide in blood samples obtained from a plurality of subjects.

28. A method for monitoring peptide levels in a subject, the method comprising:
implementing the method of claim 1 to detect the peptide in blood samples obtained from the subject at one or more points in time.

29. A method for determining hepcidin levels in a subject, the method comprising:
(a) adding a known quantity of isotopic hepcidin to a blood sample obtained from a subject, wherein the isotopic hepcidin has a molecular weight in a range of 8 Da to 12 Da greater than the molecular weight of hepcidin,
(b) detecting hepcidin and isotopic hepcidin in the blood sample by performing mass spectroscopy on the blood sample after step (a), and
(c) comparing the hepcidin and isotopic hepcidin detected in (b).

30. (canceled)

31. A method for monitoring hepcidin levels in a subject, the method comprising:
implementing the method of claim 29 to determine hepcidin levels in the subject at one or more points in time.

32-50. (canceled)

51. The method of claim 29, wherein detecting hepcidin and isotopic hepcidin in (b) comprises:
(A.) processing the blood sample of step (a) by:
(i.) combining the blood sample with a denaturation buffer comprising a volatile organic acid, wherein the concentration of the volatile organic acid in the combination is between 1% and 50%; and
(ii.) separating a soluble fraction from an insoluble fraction of the combination; and
(B.) performing MALDI-TOF mass spectrometry on the soluble fraction to detect the hepcidin and isotopic hepcidin.

52-71. (canceled)

72. An isotopic hepcidin having (a) an amino acid sequence as set forth in SEQ ID NO: 3, wherein the arginine at amino acid position 16 is arginine-¹⁵N₄¹³C₆; or
(b) an amino acid sequence as set forth in SEQ ID NO:7, wherein the phenylalanine at amino acid position 4 or 9 is phenylalanine-¹⁵N¹³C; or
(c) an amino acid sequence as set forth in SEQ ID NO: 9, wherein the phenylalanine at amino acid position 4 or 9 is phenylalanine-¹⁵N¹³C.

73-74. (canceled)

75. A composition comprising the isotopic hepcidin of claim 72.

76. (canceled)

77. A kit comprising:
a container housing isotopic hepcidin of claim 72.

78. (canceled)

79. A plate comprising a plurality of wells, each well comprising isotopic peptide having a molecular weight in a range of 8 Da to 12 Da greater than the molecular weight of a corresponding non-isotopic peptide, wherein the peptide has a molecular weight in a range of 0.2 kDa to 10 kDa.

80-83. (canceled)

84. A plate comprising a plurality of wells, each well comprising a composition comprising a volatile organic acid at a concentration in a range of 2% to 16%.

85-101. (canceled)

102. A kit comprising a container housing an isotopic peptide and
(a) a container housing a denaturation buffer comprising:
(i.) a volatile organic acid, and/or
(ii.) an organic solvent; and/or
(b) a container housing a pre-treatment solution comprising a surfactant.

103-113. (canceled)

114. A composition comprising: a volatile organic acid at a concentration in a range of 1% to 8% and an organic solvent at a concentration in a range of 5% to 20%.

115-133. (canceled)
